SEQUENCE LISTING (17 sequences)

SEQ ID NO: 1 - DNA, 2490 bp, synthetic construct
  source 1..2490: mol_type = other DNA
acatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagctccccccgctcctccccccgctcctccccccgctcctccccccgctcctccccccgctcctccccccgctcctccccccgctcctccccccgctcctccccccgctcctcccccccccgctcccgcggccccgcccccaacgcccgctcctccccccgctcccgcggccccgcccccaacgcccgccgcgcgcgcgcacgccgcccttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttaccggcgaaggagggccaccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 2 - DNA, 2752 bp, synthetic construct
  source 1..2752: mol_type = other DNA
acatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatgaaacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttaccggcgaaggagggccaccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 3 - DNA, 56 bp, synthetic construct
  source 1..56: mol_type = other DNA
aataaaggaaatttattttcattgcaatagtgtgttggaattttttgtgtctctca

SEQ ID NO: 4 - DNA, 176 bp, synthetic construct
  source 1..176: mol_type = other DNA
ctccccccgctcctccccccgctcctccccccgctcctccccccgctcctccccccgctcctccccccgctcctccccccgctcctccccccgctcctccccccgctcctcccccccccgctcccgcggccccgcccccaacgcccgctcctccccccgctcccgcggccccgccc

SEQ ID NO: 5 - DNA, 248 bp, synthetic construct
  source 1..248: mol_type = other DNA
ctccccccgctcctccccccgctcctccccccgctcctccccccgctcctccccccgctcctccccccgctcctccccccgctcctccccccgctcctccccccgctcctcccccccccgctcccgcggccccgcccccaacgcccgctcctccccccgctcccgcggccccgcccccaacgcccgccgcgcgcgcgcacgccgcccttgactcacggggatttccaagtctccaccccattgacgtc

SEQ ID NO: 6 - DNA, 160 bp, synthetic construct
  source 1..160: mol_type = other DNA
tgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 7 - DNA, 12 bp, synthetic construct
  source 1..12: mol_type = other DNA
cgctcctccccc

SEQ ID NO: 8 - DNA, 57 bp, synthetic construct
  source 1..57: mol_type = other DNA
tgactcacggggatttccaagtctccaccccattgacgtcaatgggagtttgttttg

SEQ ID NO: 9 - DNA, 100 bp, synthetic construct
  source 1..100: mol_type = other DNA
ccaaaatcaacgggactttccaaaatgtcgtaacaactccgccccattgacgcaaatgggcggtaggcgtgtacggtgggaggtctatataagcagagct

SEQ ID NO: 10 - DNA, 1610 bp, synthetic construct
  source 1..1610: mol_type = other DNA
atgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagatttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 11 - DNA, 406 bp, synthetic construct
  source 1..406: mol_type = other DNA
cgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggttgactcacggggatttccaagtctccaccccattgacgtcaatgggagtttgttttg

SEQ ID NO: 12 - DNA, 100 bp, synthetic construct; identical to SEQ ID NO: 9
  source 1..100: mol_type = other DNA

SEQ ID NO: 13 - DNA, 608 bp, synthetic construct
  source 1..608: mol_type = other DNA
gtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggasctccccccgctcctccccccgctcctccccccgctcctccccccgctcctccccccgctcctccccccgctcctccccccgctcctccccccgctcctccccccgctcctcccccccccgctcccgcggccccgcccccaacgcccgctcctccccccgctcccgcggccccgcccccaacgcccgccgcgcgcgcgcacgccgcccttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaa

SEQ ID NO: 14 - DNA, 582 bp, synthetic construct
  source 1..582: mol_type = other DNA
gtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccccccgctcctccccccgctcctccccccgctcctccccccgctcctccccccgctccycccccccccccccccccccccycccccccccscccccgcggccccgcccccaacscccgctcctccccccgctcccgcggccccgcccccaacgcccgccgcgcgcgcgcacgccgcccttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaa

SEQ ID NO: 15 - DNA, 581 bp, synthetic construct
  source 1..581: mol_type = other DNA
atcccccgctcctcccccgctcctccccccgctcctccccccgctcctccccccgctcctcccccgctcctccccccgctcctccccccgctcctccccccgctcctccccccgctcctccccccgctcctccccccgctcctccccccgctcccgcggccccgcccccaacgcccgctcctccccccgctcccgcggccccgcccccaacgcccgccgcgcgcgcgcacgccgcccggaccgccgcccgccttttttgcgcgccgccccgcccgcggggggcccgggctgcgccgccgcgctttaaagggccgcgcgcgacccccggggggtgtgtttcggggggggcccgtttctcccgctcctccccccgctcctccccccgctcctccccccgctcctccccccgctcctccccccgctcctccccccgctcctccccccgctcctccccccgctcctccccccgctcctccccccgctcctccccccgctcccgcggccccgcccccaacgcccgctcctccccccgctcccgcggccccgcccccaacgcccgccgcgcgcgcgcacgccgccct

SEQ ID NO: 16 - DNA, 464 bp, synthetic construct
  source 1..464: mol_type = other DNA
ctccccccgctcctccccccgctcctccccccgctcctccccccgctcctccccccgctcctccccccgctcctccccccgctcctccccccgctcctccccccgctcctcccccccccgctcccgcggccccgcccccaacgcccgctcctccccccgctcccgcggccccgcccccaacgcccgccgcgcgcgcgcacgccgcccttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttaccggcgaaggagggccacc

SEQ ID NO: 17 - DNA, 2075 bp, synthetic construct
  source 1..2075: mol_type = other DNA
ctccccccgctcctccccccgctcctccccccgctcctccccccgctcctccccccgctcctccccccgctcctccccccgctcctccccccgctcctccccccgctcctcccccccccgctcccgcggccccgcccccaacgcccgctcctccccccgctcccgcggccccgcccccaacgcccgccgcgcgcgcgcacgccgcccttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttaccggcgaaggagggccaccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

(19) United States
(12) Patent Application Publication
(10) Pub. No.: US 2026/0097135 A1
(43) Pub. Date: Apr. 9, 2026

(54) MODIFIED ONCOLYTIC HERPES SIMPLEX VIRUS (oHSV) AND METHODS OF USE THEREOF

(71) Applicant: The General Hospital Corporation, Boston, MA (US)

(72) Inventors: Robert L. Martuza, Cambridge, MA (US); Samuel D. Rabkin, Boston, MA (US); Dipongkor Saha, Malden, MA (US); Nusrat Jahan, Boston, MA (US)

(21) Appl. No.: 19/114,912

(22) PCT Filed: Sep. 26, 2023
(86) PCT No.: PCT/US2023/075127

§ 371 Date: Mar. 25, 2025

Related U.S. Application Data
(60) Provisional application No. 63/409,908, filed on Sep. 26, 2022.

(51) Int. Cl. A61K 48/00; A61K 38/20; A61K 45/06; C12N 15/86

(52) U.S. Cl. CPC A61K 48/0066; A61K 38/208; A61K 45/06; C12N 15/86; C12N 2710/16643; C12N 2710/16662

(57) ABSTRACT

Described herein is an oncolytic herpes simplex virus, G47ΔhIL12A, which is G47Δ containing a cassette expressing a transgene, e.g., human IL-12, driven by a spontaneously arising genetically altered HCMV immediate-early (IE) enhancer/promoter. This virus has augmented (A) production of the transgene and increased virus replication while retaining safety. Also provided are methods of use thereof for treating cancer, e.g., glioblastoma (GBM) and triple-negative breast cancer (TNBC).

### CLAIM OF PRIORITY

This application claims the benefit of U.S. Provisional Application Ser. No. 63/409,908, filed on Sep. 26, 2023. The entire contents of the foregoing are incorporated herein by reference.

### FEDERALLY SPONSORED RESEARCH OR DEVELOPMENT

This invention was made with Government support under Grant No. NS032677 awarded by the National Institutes of Health. The Government has certain rights in the invention.

### SEQUENCE LISTING

This application contains a Sequence Listing that has been submitted electronically as an XML file named 29539-0620WO1_SL_ST26.xml. The XML file, created on Sep. 26, 2023, is 28,203 bytes in size. The material in the XML file is hereby incorporated by reference in its entirety.

### TECHNICAL FIELD

Described herein are modified promoters, viral vectors comprising the promoters, and an oncolytic herpes simplex virus, G47ΔhIL12A, which is G47Δ containing a cassette expressing a transgene, e.g., human IL-12, driven by a spontaneously arising genetically altered HCMV immediate-early (IE) enhancer/promoter. This virus has augmented (A) production of the transgene and increased virus replication while retaining safety. Also provided are methods of use thereof, e.g., for treating cancer, e.g., glioblastoma (GBM) and triple-negative breast cancer (TNBC).

### BACKGROUND

The first genetically engineered oncolytic virus, dlsptk, was described in 1991 [1]. Although that virus was not appropriate for clinical use, it provided proof of concept leading to the development of the next virus, G207 [2], which was the first oncolytic virus (OV) taken into clinical trial for GBM [3]. G207 is still in use and has shown efficacy in pediatric malignant glioma [4]. The next iteration was G47Δ [5]. This was an improvement over G207 in that it replicated better in glioblastoma stem-like cells (GSC) [6, 7] and the deletion of ICP47 overcame the inhibition of MHC class I presentation setting the stage for improved oncolytic immunovirotherapy [5]. Two intratumoral inoculations of G47Δ in patients with recurrent GBM achieved a 1-year survival rate of 38.5% [8], while up to 6 inoculations of G47Δ increased 1-year survival from a historic 15% to 92% [9], leading to approval by the Japanese Ministry of Health Labor, and Welfare [10].

### SUMMARY

Provided herein are expression cassettes comprising an enhancer sequence comprising two or more DR2 repeat sequence units (CGCTCCTCCCCC (SEQ ID NO:7) inserted upstream of a promoter, preferably within 500, 250, 200, or 100 nucleotides of the promoter, and optionally a transgene operably linked to the promoter. In some embodiments, the enhancer sequence comprises from six to 24 DR2 repeat sequence units (CGCTCCTCCCCC (SEQ ID NO:7). In some embodiments, the enhancer sequence comprises

(SEQ ID NO: 4)
CTCCCCCCGCTCCTCCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCTCCCCCCCCCGCTCCCGCGGCCCCGC
CCCCAACGCCCGCTCCTCCCCCCGCTCCCGCGGCCCCGCCC
or
(SEQ ID NO: 5)
CTCCCCCCGCTCCTCCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCTCCCCCCCCCGCTCCCGCGGCCCCGC
CCCCAACGCCCGCTCCTCCCCCCGCTCCCGCGGCCCCGCCCCCAA
CGCCCGCCGCGCGCGCGCACGCCGCCCTTGACTCACGGGGATTTC
CAAGTCTCCACCCCATTGACGTC
or
(SEQ ID NO: 15)
ATCCCCCGCTCCTCCCCCGCTCCTCCCCCCGCTCCTCCCCCCGCT
CCTCCCCCCGCTCCTCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCCGCGGCCCCGCCCCCAACGCCCGCTC
CTCCCCCCGCTCCCGCGGCCCCGCCCCCAACGCCCGCCGCGCGCG
CGCACGCCGCCCGGACCGCCGCCCGCCTTTTTTGCGCGCCGCCCC
GCCCGCGGGGGGCCCGGGCTGCGCCGCCGCGCTTTAAAGGGCCGC
GCGCGACCCCCGGGGGGTGTGTTTCGGGGGGGGCCCGTTTCTCCC
GCTCCTCCCCCCGCTCCTCCCCCCGCTCCTCCCCCCGCTCCTCCC
CCCGCTCCTCCCCCCGCTCCTCCCCCCGCTCCTCCCCCCGCTCCT
CCCCCCGCTCCTCCCCCCGCTCCTCCCCCCGCTCCTCCCCCCGCT
CCCGCGGCCCCGCCCCCAACGCCCGCTCCTCCCCCCGCTCCCGCG
GCCCCGCCCCCAACGCCCGCCGCGCGCGCGCACGCCGCCCT.

In some embodiments, the expression cassette is in a plasmid, cosmid, or viral vector. In some embodiments, the viral vector is selected from the group consisting of recombinant retroviruses, adenovirus, adeno-associated virus, alphavirus, oncoviruses, and lentiviruses. In some embodiments, the oncovirus is a herpes simplex virus. In some embodiments, the herpes simplex virus comprises one or more of: one or more alterations selected from deletion of both copies of γ34.5; deletion of ICP47 and/or Us11 promoter; an inactivating insertion in ICP6; downregulation, tumor-selective expression, and/or specific amino acid mutations of γ34.5, ICP47, and/or ICP6; upregulation or tumor-selective expression of Us11; and a reporter gene like GFP or LacZ. In some embodiments, the expression cassette is in G207, MG18L, Δ68H-6, or G47Δ.

In some embodiments, the expression cassette comprises a transgene, optionally wherein the transgene is selected from cytokines, chemokines, complement components and their receptors, immune system accessory molecules, adhesion molecules, adhesion receptor molecules, and adenosine-degrading enzymes. In some embodiments, the cytokine is selected from interleukins, optionally any of interleukins 1-15, preferably IL-12; α, β, or γ-interferons; tumor necrosis factor; granulocyte macrophage colony stimulating factor (GM-CSF); macrophage colony stimulating factor (M-CSF); and granulocyte colony stimulating factor (G-CSF). In some embodiments, the chemokine is selected from B7.1 and B7.2. In some embodiments, the adhesion molecule is selected from ICAM-1, 2, and 3. In some embodiments, the immune system accessory molecule is selected from neutrophil activating protein (NAP), macrophage chemoattractant and activating factor (MCAF), RANTES, and macrophage inflammatory peptides MTP-1a and MTP-1b.

Also provided herein are methods of expressing a transgene in a cell, the method comprising contacting the cell with an expression cassette as described herein, wherein the expression cassette comprises the transgene.

Additionally, provided herein are oncolytic herpes simplex virus (oHSV) comprising a promoter and an enhancer sequence, wherein the enhancer sequence comprises from six to 24 DR2 repeat sequence units (CGCTCCTCCCCC (SEQ ID NO:7), preferably wherein the enhancer sequence comprises

(SEQ ID NO: 4)
CTCCCCCCGCTCCTCCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCTCCCCCCCCCGCTCCCGCGGCCCCGC
CCCCAACGCCCGCTCCTCCCCCCGCTCCCGCGGCCCCGCCC
or
(SEQ ID NO: 5)
CTCCCCCCGCTCCTCCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCTCCCCCCCCCGCTCCCGCGGCCCCGC
CCCCAACGCCCGCTCCTCCCCCCGCTCCCGCGGCCCCGCCCCCAA
CGCCCGCCGCGCGCGCGCACGCCGCCCTTGACTCACGGGGATTTC
CAAGTCTCCACCCCATTGACGTC
or
(SEQ ID NO: 15)
ATCCCCCGCTCCTCCCCCGCTCCTCCCCCCGCTCCTCCCCCCGCT
CCTCCCCCCGCTCCTCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCCGCGGCCCCGCCCCCAACGCCCGCTC
CTCCCCCCGCTCCCGCGGCCCCGCCCCCAACGCCCGCCGCGCGCG
CGCACGCCGCCCGGACCGCCGCCCGCCTTTTTTGCGCGCCGCCCC
GCCCGCGGGGGGCCCGGGCTGCGCCGCCGCGCTTTAAAGGGCCGC
GCGCGACCCCCGGGGGGTGTGTTTCGGGGGGGGCCCGTTTCTCCC
GCTCCTCCCCCCGCTCCTCCCCCCGCTCCTCCCCCCGCTCCTCCC
CCCGCTCCTCCCCCCGCTCCTCCCCCCGCTCCTCCCCCCGCTCCT
CCCCCCGCTCCTCCCCCCGCTCCTCCCCCCGCTCCTCCCCCCGCT
CCCGCGGCCCCGCCCCCAACGCCCGCTCCTCCCCCCGCTCCCGCG
GCCCCGCCCCCAACGCCCGCCGCGCGCGCGCACGCCGCCCT.

In some embodiments, the oHSV further comprises a transgene, optionally wherein the transgene is selected from cytokines, chemokines, complement components and their receptors, immune system accessory molecules, adhesion molecules, adhesion receptor molecules, and adenosine-degrading enzymes. In some embodiments, the cytokine is selected from interleukins, optionally any of interleukins 1-15; α, β, or γ-interferons; tumor necrosis factor; granulocyte macrophage colony stimulating factor (GM-CSF); macrophage colony stimulating factor (M-CSF); and granulocyte colony stimulating factor (G-CSF). In some embodiments, the transgene is human interleukin 12 (hIL-12). In some embodiments, the chemokine is selected from B7.1 and B7.2. In some embodiments, the adhesion molecule is selected from ICAM-1, -2, and -3. In some embodiments, the immune system accessory molecule is selected from neutrophil activating protein (NAP), macrophage chemoattractant and activating factor (MCAF), RANTES, and macrophage inflammatory peptides MTP-1a and MTP-1b.

In some embodiments, the oHSV comprises (i) a mutation that consists of a deletion of a region corresponding to the BstEII-EcoNI fragment of the BamHI x fragment of F strain of herpes simplex virus I, and (ii) an inactivating mutation in a γ 34.5 neurovirulence locus, and optionally (iii) optionally an inactivating mutation elsewhere in the genome and/or (iv) a reporter gene like GFP or LacZ.

In some embodiments, the oHSV is G47Δ.

Additionally, provided herein are oncolytic herpes simplex virus (oHSV) comprising a promoter and an enhancer sequence comprising:

(SEQ ID NO: 16)
CTCCCCCCGCTCCTCCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCTCCCCCCCCCGCTCCCGCGGCCCCGC
CCCCAACGCCCGCTCCTCCCCCCGCTCCCGCGGCCCCGCCCCCAA
CGCCCGCCGCGCGCGCGCACGCCGCCCTTGACTCACGGGGATTTC
CAAGTCTCCACCCCATTGACGTCAATGGGAGTTTGTTTTGGCACC
AAAATCAACGGGACTTTCCAAAATGTCGTAACAACTCCGCCCCAT
TGACGCAAATGGGCGGTAGGCGTGTACGGTGGGAGGTCTATATAA
GCAGAGCTCTCTGGCTAACTAGAGAACCCACTGCTTACTGGCTTA
TCGAAATTAATACGACTCACTATAGGGAGACCCAAGCTTACCGGC
GAAGGAGGGCCACC.

In some embodiments, the oHSV comprises (i) a mutation that consists of a deletion of a region corresponding to the BstEII-EcoNI fragment of the BamHI x fragment of F strain of herpes simplex virus I, and (ii) an inactivating mutation in a γ 34.5 neurovirulence locus, and optionally (iii) optionally an inactivating mutation elsewhere in the genome and/or (iv) a reporter gene like GFP or LacZ. In some embodiments, the oHSV is G47Δ. In some embodiments, the oHSV comprises a human IL-12 transgene operably linked to the promoter and enhancer of SEQ ID NO:12.

Also provided herein are oHSV comprising a human IL-12 expression cassette comprising SEQ ID NO:17, optionally wherein the oHSV is G47Δ.

Further, provided herein are methods of treating a cancer in a subject, the method comprising administering to the subject an effective amount of an oHSV as described herein. In some embodiments, the cancer is a solid tumor. In some embodiments, the solid tumor is a nervous system tumor, optionally an astrocytoma, oligodendroglioma, meningioma, neurofibroma, glioma, glioblastoma, ependymoma, schwannoma, neurofibrosarcoma, neuroblastoma, pituitary tumor, or medulloblastoma. In some embodiments, the nervous system tumor is a glioblastoma. In some embodiments, the solid tumor is melanoma, prostate carcinoma, renal cell carcinoma, pancreatic cancer, breast cancer, lung cancer, colon cancer, gastric cancer, fibrosarcoma, squamous cell carcinoma, neurectodermal, thyroid tumor, lymphoma, hepatoma, mesothelioma, or epidermoid carcinoma cells. In some embodiments, the breast cancer is triple negative breast cancer.

In some embodiments, the methods further comprise administering an immunomodulator, optionally comprising one or more immune checkpoint inhibitors (ICIs) and/or inhibitors of the adenosine pathway, e.g., small molecule inhibitors and antibodies to CD39, CD73, A2a Receptor (A2aR), and A2bR. In some embodiments, the one or more ICIs are selected from an antibody that binds to PD-1, CD40, PD-L1, Tim3, Lag3, CTLA-4, or T-cell immunoglobulin and ITIM domains (TIGIT). In some embodiments, the one or more ICIs comprise an anti-PD-1 antibody and an anti-CTLA-4 antibody.

Also provided herein are methods of treating cancer, e.g., glioblastoma, in a subject comprising administering to the subject an oncolytic herpes simplex virus (oHSV) comprising a promoter and an enhancer sequence, wherein the enhancer sequence comprises from six to 24 DR2 repeat sequence units (CGCTCCTCCCCC (SEQ ID NO:7), preferably wherein the enhancer and promoter sequence comprises SEQ ID NO:16, in combination with an anti-PD-1 antibody and an anti-CTLA-4 antibody. In some embodiments, the oHSV is G207, G207-Us11, Δ68H-6, or MG18L. In some embodiments, the oHSV comprises SEQ ID NO:1.

Additionally, provided herein are methods of treating cancer, e.g., glioblastoma, in a subject comprising administering to the subject a G47Δ oncolytic herpes simplex virus (oHSV) comprising a promoter and an enhancer sequence, wherein the enhancer sequence comprises from six to 24 DR2 repeat sequence units (CGCTCCTCCCCC (SEQ ID NO:7), preferably wherein the enhancer and promoter sequence comprises SEQ ID NO:16, in combination with an anti-PD-1 antibody and an anti-CTLA-4 antibody. In some embodiments, the oHSV is G47ΔhIL12A. In some embodiments, the oHSV comprises SEQ ID NO:1 or a sequence at least 80%, 90%, 95%, or 99% identical to nucleotides 93-2490 of SEQ ID NO:1, or to nucleotides 416-2490 of SEQ ID NO:1.

Further, provided herein are compositions comprising an oncolytic herpes simplex virus (oHSV) comprising a promoter and an enhancer sequence, wherein the enhancer sequence comprises from six to 24 DR2 repeat sequence units (CGCTCCTCCCCC (SEQ ID NO:7), preferably wherein the enhancer and promoter sequence comprises SEQ ID NO:16, in combination with an anti-PD-1 antibody and an anti-CTLA-4 antibody. In some embodiments, the oHSV is G207, G207-Us11, Δ68H-6, or MG18L. In some embodiments, the oHSV comprises SEQ ID NO:1 or a sequence at least 80%, 90%, 95%, or 99% identical to nucleotides 93-2490 of SEQ ID NO:1, or to nucleotides 416-2490 of SEQ ID NO:1.

In some embodiments, the compositions described herein are provided for use in methods of treating cancer as described herein.

Provided herein are compositions comprising a genetically-engineered oncolytic HSV expressing human hIL12 from a novel transcriptional regulatory region containing a hybrid promoter with the HCMV IE enhancer replaced with HSV repeat ‘a’ sequences. In some embodiments, the composition is constructed from oncolytic HSV G47Δ (γ34.5 deleted, ICP47 deleted, UL39 (ICP6) inactivated) and expressing hIL-12. In some embodiments, the hIL-12 cassette is inserted into the ICP6 gene. In some embodiments, the hIL-12 cassette is inserted at a different site in the HSV-1 genome.

In some embodiments, G47ΔhIL12A replicates better and expresses more hIL-12 than a recombinant isolate without the deletion/insertion of claim 1.

In some embodiments, G47ΔhIL12A does not express eGFP.

In some embodiments, the hIL-12 cassette is inserted at the same site in other oncolytic HSVs, for example, MG18L, G207, G207-Us 11, Δ68H-6, etc. In some embodiments, the hIL-12 cassette is inserted at a different site in other oncolytic HSVs, for example, MG18L, G207, G207-Us 11, Δ68H-6, etc.

In some embodiments, the oncolytic HSV replicates in glioblastoma stem-like cells and is safe in the brain.

In some embodiments, the oncolytic HSV is sensitive to antiviral nucleoside analog inhibitors, such as acyclovir.

In some embodiments, hIL-12 sequences are replaced with other therapeutic transgenes, such as cytokines, chemokines, immune checkpoint inhibitors, other immune modulators, anti-angiogenesis factors, adenosine degrading enzymes, etc.

Also provided herein are methods to prevent, treat, or slow the progression of cancer comprised of administering to a patient, one or multiple times, therapeutically effective amounts of a composition described herein. Additionally provided are methods to prevent, treat, or slow the progression of cancer comprised of administering to a patient, one or multiple times, therapeutically effective amounts of the composition described herein and two or more immune checkpoint inhibitors, termed triple combination therapy. Further, provided herein is the use of a composition described herein, wherein the oncolytic HSV is combined with other pharmaceutical agents, such as immune checkpoint inhibitors, immune co-stimulatory agonists, DNA damage response inhibitors, small molecule inhibitors, chemotherapy, radiation therapy, molecularly targeted drugs, adenosine pathway inhibitors, etc. Additionally, provided herein is the use of a composition described herein, wherein the oncolytic HSV is combined with other biological agents, such as CAR-T cells, BiTEs, immune cells, vaccines, etc.

In some embodiments, the cancer is a solid tumor. In some embodiments, the solid tumor resides in the nervous system and includes, but is not limited to, glioblastoma. In some embodiments, the solid tumor resides outside the nervous system, for example, breast cancer, prostate cancer, lung cancer, squamous cell carcinoma, sarcoma, colon cancer, skin cancers, pancreatic cancer, etc. In some embodiments, the solid tumor is metastatic to the brain. In some embodiments, the solid tumor is metastatic to organs outside the brain.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-B are schematic representation of an exemplary G47ΔhIL12 construct and exemplary transfer plasmid used to construct G47ΔhIL12, showing the genetic alterations occurring in G47ΔhIL12. FIG. 1A depicts the genomic structure of an exemplary G47ΔhIL12 (line 1), which is derived from G47Δ, containing deletions of both copies of the γ34.5 gene (each ˜1 kb), a 312 bp deletion of the ICP47 gene, as indicated by the Δ symbols, and an ICP6 gene inactivation due to the insertion of a cassette expressing eGFP and human IL-12 (hIL-12). The HSV genome consists of long and unique short sequences (U_{L} and U_{S}) bracketed by terminal (TR_{L} and TR_{S}) and internal (IR_{L} and IR_{S}) inverted repeat sequences. The ‘a’ sequence repeats [reiteration Ib, CCCGCTCCTCCC (SEQ ID NO:7); GenBank: J02223.1] are at the termini of the repeat regions. Lines 2 and 3 illustrate the sequence arrangements of the inserted hIL-12 (p40 and p35 subunit) cassettes. G47ΔhIL12B (line 2) and G47ΔhIL12A (line 3) represent the 2 distinct isolates. G47ΔhIL12B contains the complete sequence inserted from pNJ-ICP6-eGFP-CMV-hIL12, as expected. A novel feature of G47ΔhIL12A is the deletion of the HCMV IE enhancer [14] and insertion of HSV terminal repeat ‘a’ sequences upstream of IL-12. FIG. 1B depicts the annotated map of the hIL12 transfer plasmid pNJ-ICP6-eGFP-CMV-hIL12 that was used to generate G47ΔhIL12, which contains the sequences to be inserted bracketed by ICP6.

FIGS. 2A-B show the human IL-12 levels in supernatants from Vero cells infected with G47ΔhIL12 isolates, multiplicity of infection (MOI)=1 at 24 hours post-infection (hpi). FIG. 2A depicts the hIL-12 levels in supernatants from twice plaque-purified independent virus isolates. G47ΔhIL12 isolate 10-3-A11H4H4 (hatched box) is G47ΔhIL12A and 8-1B4A3D9 (grey box) is G47ΔhIL12B. FIG. 2B is a graph of the hIL-12 levels for the three G47ΔhIL12 isolates that produced the highest (filled circles) and lowest hIL-12 (filled squares) levels at 24 hpi of Vero cells at MOI=1. *** p=0.0003.

FIGS. 3A-C depict the sequence of the region upstream of hIL-12. FIGS. 3A-C are the sequences of viral DNA from the two G47ΔhIL12 isolates (A and B, respectively), obtained by next-generation sequencing (NGS; G47ΔhIL12A and G47ΔhIL12B (SEQ ID NOs:1 and 2, respectively)) and (C) NGS and PacBio Sequel II long-read sequencing (G47ΔhIL12A; SEQ ID NOs: 13 and 14) of viral DNA. 3A, The sequence features of G47ΔhIL12A from the top were as follows. The β-globin polyA sequence after GFP (FIGS. 1A-B) is indicated with lower case font (aataaaggaaatttattttcattgcaatagtgtgttggaattttttgtgtctctca, SEQ ID NO:3). In G47ΔhIL12A (left panel), an HSV repeat sequence (CTCCCCCCGCTCCTCCCCCCGCTCCTCCCCCCGCTCCTCCCCCCGCTCCTC CCCCCGCTCCTCCCCCCGCTCCTCCCCCCGCTCCTCCCCCCGCTCCTCCCCC CGCTCCTCCCCCCCCCGCTCCCGCGGCCCCGCCCCCAACGCCCGCTCCTCC CCCCGCTCCCGCGGCCCCGCCC, SEQ ID NO:4, bold and double underlined) is inserted as part of a larger sequence (CTCCCCCCGCTCCTCCCCCCGCTCCTCCCCCCGCTCCTCCCCCCGCTCCTC CCCCCGCTCCTCCCCCCGCTCCTCCCCCCGCTCCTCCCCCCGCTCCTCCCCC CGCTCCTCCCCCCCCCGCTCCCGCGGCCCCGCCCCCAACGCCCGCTCCTCC CCCCGCTCCCGCGGCCCCGCCCCCAACGCCCGCCGCGCGCGCGCACGCCG CCCT, SEQ ID NO:5, double underlined) that is not present in pNJ-ICP6-eGFP-CMV-hIL12. The HSV repeat sequence has homology to HSV strain F sequences (GenBank GU734771.1) from TR_{L} (151435-151608) or IRs (reiteration set 1; 12434-126572), as well as 10 copies of the DR2 repeat sequence unit (CGCTCCTCCCCC (SEQ ID NO:7); GenBank J02223.1, Mocarski and Roizman, Proc Natl Acad Sci USA. 1981 November; 78(11):7047-51). This repeat-containing sequence is inserted upstream of a sequence fragment from the HCMV IE enhancer and the HCMV IE promoter (TGACTCACGGGGATTTCCAAGTCTCCACCCCATTGACGTCAATGGGAGTT TGTTTTGGCACCAAAATCAACGGGACTTTCCAAAATGTCGTAACAACTCC GCCCCATTGACGCAAATGGGCGGTAGGCGTGTACGGTGGGAGGTCTATAT AAGCAGAGCT (SEQ ID NO:6)), which includes a short truncated sequence from the HCMV IE enhancer (TGACTCACGGGGATTTCCAAGTCTCCACCCCATTGACGTCAATGGGAGTTTGTT TTG (italic font), SEQ ID NO:8) [14] upstream of the HCMV IE promoter (Bold font, CCAAAATCAACGGGACTTTCCAAAATGTCGTAACAACTCCGCCCCATT GACGCAAATGGGCGGTAGGCGTGTACGGTGGGAGGTCTATATAAGCA GAGCT (SEQ ID NO:9) sequences, which drives human IL-12 p40 and p35 intiated from ATG (bold italic font) separated by a linker sequence (lower case) sequence shown, ATGGGTCACCAGCAGTTGGTCATCTCTTGGTTTTCCCTGGTTTTTCTGGCAT CTCCCCTCGTGGCCATATGGGAACTGAAGAAAGATGTTTATGTCGTAGAAT TGGATTGGTATCCGGATGCCCCTGGAGAAATGGTGGTCCTCACCTGTGAC ACCCCTGAAGAAGATGGTATCACCTGGACCTTGGACCAGAGCAGTGAGGT CTTAGGCTCTGGCAAAACCCTGACCATCCAAGTCAAAGATTTGGAGATGC TGGCCAGTACACCTGTCACAAAGGAGGCGAGGTTCTAAGCCATTCGCTCC TGCTGCTTCACAAAAAGGAAGATGGAATTTGGTCCACTGATATTTTAAAG GACCAGAAAGAACCCAAAAATAAGACCTTTCTAAGATGCGAGGCCAAGA ATTATTCTGGACGTTTCACCTGCTGGTGGCTGACGACAATCAGTACTGATT TGACATTCAGTGTCAAAAGCAGCAGAGGCTCTTCTGACCCCCAAGGGGTG ACGTGCGGAGCTGCTACACTCTCTGCAGAGAGAGTCAGAGGGGACAACA AGGAGTATGAGTACTCAGTGGAGTGCCAGGAGGACAGTGCCTGCCCAGCT GCTGAGGAGAGTCTGCCCATTGAGGTCATGGTGGATGCCGTTCACAAGCT CAAGTATGAAAACTACACCAGCAGCTTCTTCATCAGGGACATCATCAAAC CTGACCCACCCAAGAACTTGCAGCTGAAGCCATTAAAGAATTCTCGGCAG GTGGAGGTCAGCTGGGAGTACCCTGACACCTGGAGTACTCCACATTCCTA CTTCTCCCTGACATTCTGCGTTCAGGTCCAGGGCAAGAGCAAGAGAGAAA AGAAAGATAGAGTCTTCACGGACAAGACCTCAGCCACGGTCATCTGCCGC AAAAATGCCAGCATTAGCGTGCGGGCCCAGGACCGCTACTATAGCTCATC TTGGAGCGAATGGGCATCTGTGCCCTGCAGTgttcctggagtaggggtacctggggtgggc GCCAGAAACCTCCCCGTGGCCACTCCAGACCCAGGAATGTTCCCATGCCT TCACCACTCCCAAAACCTGCTGAGGGCCGTCAGCAACATGCTCCAGAAGG CCAGACAAACTCTAGAATTTTACCCTTGCACTTCTGAAGAGATTGATCATG AAGATATCACAAAAGATAAAACCAGCACAGTGGAGGCCTGTTTACCATTG GAATTAACCAAGAATGAGAGTTGCCTAAATTCCAGAGAGACCTCTTTCAT AACTAATGGGAGTTGCCTGGCCTCCAGAAAGACCTCTTTTATGATGGCCCT GTGCCTTAGTAGTATTTATGAAGACTTGAAGATGTACCAGGTGGAGTTCA AGACCATGAATGCAAAGCTGCTGATGGATCCTAAGAGGCAGATCTTTCTA GATCAAAACATGCTGGCAGTTATTGATGAGCTGATGCAGGCCCTGAATTT CAACAGTGAGACTGTGCCACAAAAATCCTCCCTTGAAGAACCGGATTTTT ATAAAACTAAAATCAAGCTCTGCATACTTCTTCATGCTTTCAGAATTCGGG CAGTGACTATTGATAGAGTGATGAGCTATCTGAATGCTTCCTAA; SEQ ID NO:10). 3B, The sequence recombined into G47ΔhIL12B was the same as that in plasmid pNJ-ICP6-eGFP-CMV-hIL12 used for recombination. It contains the complete HCMV IE enhancer (italic font) (GenBank K03104.1; CGCGTTACATAACTTACGGTAAATGGCCCGCCTGGCTGACCGCCCAACGACCC CCGCCCATTGACGTCAATAATGACGTATGTTCCCATAGTAACGCCAATAGGGACT TTCCATTGACGTCAATGGGTGGAGTATTTACGGTAAACTGCCCACTTGGCAGTAC ATCAAGTGTATCATATGCCAAGTACGCCCCCTATTGACGTCAATGACGGTAAATG GCCCGCCTGGCATTATGCCCAGTACATGACCTTATGGGACTTTCCTACTTGGCA GTACATCTACGTATTAGTCATCGCTATTACCATGGTGATGCGGTTTTGGCAGTACA TCAATGGGCGTGGATAGCGGTTGACTCACGGGGATTTCCAAGTCTCCACCCCA TTGACGTCAATGGGAGTTTGTTTTG, SEQ ID NO:11) and promoter (Bold font, CCAAAATCAACGGGACTTTCCAAAATGTCGTAACAACTCCGCCCCATT GACGCAAATGGGCGGTAGGCGTGTACGGTGGGAGGTCTATATAAGCA GAGCT, SEQ ID NO:12), which drives human IL-12 p40 and p35 following ATG (Bold italic font, SEQ ID NO:10). The sequence deleted in G47ΔhIL12A has font [symbol] [symbol] FIG. 3C is a BLASTn (National Library of Medicine) sequence comparison between the NGS sequence (SEQ ID NO:13) and PacBio sequence of G47ΔhIL12A (SEQ ID NO:14). S=G or C and Y=C or T. The DR2 repeat sequences (CGCTCCTCCCCC, SEQ ID NO:7; GenBank J02223.1) have font

[symbol]

FIG. 4 shows a gel of a restriction endonuclease digest of G47ΔhIL12 DNAs. Viral DNAs from; G47Δ, G47ΔhIL12A and 6-2-A8G1G6H4 (high hIL-12), and G47ΔhIL12B and 8-1-E2A11E11 (low hIL-12) were digested with BglII (left) and HindIII (right) and the digested fragments separated in a 0.6% agarose gel in Tris-acetate-EDTA buffer. The DNA fragments unique to the two viral isolates are indicated with their sizes. A BglII 2578 bp DNA fragment was only observed with G47ΔhIL12B and 8-1E2A11E11 DNAs, while a BglII 2198 bp DNA fragment was observed for all G47ΔhIL12 DNAs. A HindIII 8316 bp DNA fragment was only observed for G47ΔhIL12B and 8-1E2A11E11 clones, while a HindIII 4410 bp DNA fragment was observed for all G47ΔhIL12 DNAs. None of these DNA fragments were present with G47Δ DNA. The DNA ladders (center lanes) from left to right were 1-kb DNA ladder (Invitrogen), high-molecular-weight DNA marker (Invitrogen), and GeneRuler High Range DNA Ladder (ThermoFisher Scientific).

FIGS. 5A-C illustrate the in vitro characteristics of the two G47ΔhIL12 isolates. Vero cells (5×10⁴) in 24-well plates were infected with each virus in triplicate and incubated at 37° C. Samples were collected at indicated times and assayed for virus yield by plaque assay (FIG. 5A), for acyclovir sensitivity by plaque reduction assay (FIG. 5B), and for hIL-12 production by ELISA assay at 24 hpi (FIG. 5C). G47ΔhIL12A grows better in Vero cells than G47ΔhIL12B (FIG. 5A. p=0.0001 (***) at 48 hpi; two-tailed unpaired t-test). G47ΔhIL12A did not alter the growth properties of the virus compared to parental G47Δ, as opposed to G47ΔhIL12B, which replicated less well. G47ΔhIL12A is similarly sensitive to acyclovir (IC50=0.22 μM) as parental G47Δ (IC50=0.28 μM). Nonlinear regression curves were plotted and IC50s calculated by Prism. Error bars are the mean±SD. (FIG. 5B) and expressed more hIL-12 than G47ΔhIL12B (FIG. 5C; p=0.0005 (***) two-tailed unpaired t-test). This illustrates that the transgene construct did not alter the sensitivity of the virus to a clinically relevant anti-herpetic therapeutic yet expressed high levels of hIL-12, indeed higher than G47ΔhIL12B. All these properties are important for therapeutic efficacy.

FIGS. 6A-B are a series of graphs showing the biological activity of virus-expressed hIL-12. FIG. 6A is a graph showing the concentration of hIL-12 in Vero cell supernatants after infection with two G47ΔhIL12A isolates ((A and 10-2-A1B5G3; MOI=1.5, at 64 hp) that were used for hIL-12 bioactivity assays in FIG. 5B. FIG. 6B shows that hIL-12 expressed by G47ΔhIL12A-infected Vero cells induces human NK-92MI cells to produce IFNγ in a dose-dependent fashion. Heparin (10 μg/ml) synergizes with hIL-12 in stimulating hIFNγ production by NK-92MI cells, comparable to recombinant hIL-12 (R-hIL-12). This demonstrates that the virus-produced hIL-12 has the same biological activity as recombinant protein on human target cells. Error bars are the mean±SD.

FIGS. 7A-B depict oHSV growth in four human glioblastoma stem-like cell (GSC) lines derived from 4 patients [15, 16]. Human GSCs (20,000 per well) in 48-well plates were infected with G47Δ or G47ΔhIL12A and the cells and media were collected at the indicated times for titration of virus by plaque assay. FIG. 7A depicts multi-step growth kinetics after infection with MOI=0.3. FIG. 7B depicts single-step growth kinetics after infection with MOI=1.5. Virus yield of G47ΔhIL12A was similar to parental G47Δ at both low and high MOI. This illustrates that the transgene construct does not alter the growth properties of the virus in a therapeutic target, human GSCs.

FIG. 8 depicts the cytotoxicity of oHSV in four GSC lines. Human GSCs were infected with increasing MOI of G47Δ or G47ΔhIL12A and incubated for 4 days. MTS assay was used to determine cell viability. G47ΔhIL12A was as cytotoxic to human GSCs as parental G47Δ, having the same IC50 (indicated IC50's are for G47Δ). Error bars are the mean±SD. Nonlinear regression curves were plotted and IC50s calculated by Prism. This illustrates that the transgene construct did not alter the cytotoxic activity of G47ΔhIL12A in infected human GSCs.

FIGS. 9A-B show virus growth and production of hIL-12 in G47ΔhIL12A-infected human GSCs (MOI=1.5 at 30 hpi). FIG. 9A is a graph showing the virus yield as determined by plaque assay after harvest of cells and media. There was no significant difference in virus yield between G47ΔhIL12A and G47Δ. FIG. 9B is a graph showing the concentration of hIL-12 in collected media and determined by hIL-12 ELISA assay. Error bars are the mean±SD. Human GSCs express high levels of hIL-12 after infection with G47ΔhIL12A that doesn't directly correlate with virus yield.

FIGS. 10A-C are a series of graphs showing the safety of G47ΔhIL12A after intracerebral injection in HSV-sensitive mice. BALB/c female mice (8 weeks old) were inoculated intracerebrally with PBS, G47ΔhIL12A (2×10⁶ PFU), or parental HSV-1 wild-type F strain (1×10³ PFU or 1×10⁴ PFU) and checked daily initially and 3 times/wk after 10 days. FIG. 10A is a Kaplan Meier plot of mouse survival after virus injection. FIG. 10B is a graph showing the percent changes in body weight after virus injection. FIG. 10C depicts the changes in neurological score for each individual mouse in a group; based on general appearance (A), spontaneous activity (S), and response to external stimuli or neurological deficits (R). A, S, and R were scored 1 for severely impaired, 2 for moderately impaired, 3 for slightly impaired, and 4 for normal, with a normal score of 12 and a moribund score of 3. As opposed to the rapid decline in body weight and neurologic score with the two low doses of wild-type HSV (parental strain F), high dose G47ΔhIL12A only caused minor transient neurologic effects, and all injected mice survived long-term (100 days) in a healthy state. Thus G47ΔhIL12A is safe at a dose more than 3 logs greater than strain F, with a similar safety profile as its parent G47Δ, which has been safely injected intracerebrally into human glioblastoma patients [9].

### DETAILED DESCRIPTION

The present invention is based, at least in part, on development of an expression cassette with a genetically altered upstream enhancer sequence driving expression of a transgene within a gene delivery vehicle such as a viral genome. This sequence was developed during generation of a human interleukin 12 (hIL-12) expressing oncolytic virus. As shown herein, a hIL-12 expression cassette in a transfer plasmid was made that contained an HCMV IE enhancer/promoter, a constitutive strong promoter often used for gene expression in HSV vectors [22-26]. However, the hIL-12 expression cassette inserted into G47Δ at the LacZ insertion site in ICP6 was genetically altered by a deletion in the HCMV IE enhancer and insertion of viral ‘a’ sequences; without wishing to be bound by theory, the mutation likely occurred due to selection during recombination and the screening scheme. As shown herein, virus with a mutated promoter/enhancer sequence (one example of which is referred to herein as G47ΔhIL12A) expressed higher levels of hIL-12 and replicated better than a similar virus with an intact hIL-12 expression cassette (G47ΔhIL12B). This altered upstream sequence can be used to drive expression of any operably linked transgene, e.g., a transgene in a viral genome (including a transgene inserted at ICP6 or other locations of an HSV). As demonstrated with hIL-12, expression of the inserted sequence does not alter the replication or cytotoxicity of this virus in human glioblastoma stem-like cells (GSCs), an important target for oncolytic GBM therapy.

### Modified Enhancer Sequences and Expression Constructs

Thus, described herein are enhancer sequences and expression constructs comprising the enhancer and a promoter, e.g., viruses, plasmids, and other nucleic acids. The enhancers comprise an insertion of viral ‘a’ sequences comprising two or more, e.g., 2, 4, 6, 8, 10, 12, 14, 16, 18, 20, 22, 24, or more, e.g., 6 to 24, DR2 repeat sequence units (CGCTCCTCCCCC (SEQ ID NO:7) and further can comprise part of HCMV IE enhancer sequence (optionally, comprise no HCMV IE enhancer sequence or comprise part or all of a truncated sequence from the HCMV IE enhancer (e.g., up to 15%, 20%, 30%, 40%, 50%, 60%, 70% or 80% of SEQ ID NO:11, e.g., comprising all or part of SEQ ID NO:8 (e.g., 50%, 60%, 70%, 80%, 90%, or 95% of full length of TGACTCACGGGGATTTCCAAGTCTCCACCCCATTGACGTCAATGGGAGTTT GTTTTGGCA (SEQ ID NO:8), e.g., comprising one, two, or all three of an SP1, a CREB, and an NFkB site. In some embodiments, the enhancer comprises an HSV repeat sequence that is at least 90%, 95%, or 99% identical to

(SEQ ID NO: 4)
CTCCCCCCGCTCCTCCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCTCCCCCCCCCGCTCCCGCGGCCCCGC
CCCCAACGCCCGCTCCTCCCCCCGCTCCCGCGGCCCCGCCC
or
(SEQ ID NO: 5)
(CTCCCCCCGCTCCTCCCCCCGCTCCTCCCCCCGCTCCTCCCCCC
GCTCCTCCCCCCGCTCCTCCCCCCGCTCCTCCCCCCGCTCCTCCC
CCCGCTCCTCCCCCCGCTCCTCCCCCCCCCGCTCCCGCGGCCCCG
CCCCCAACGCCCGCTCCTCCCCCCGCTCCCGCGGCCCCGCCCCCA
ACGCCCGCCGCGCGCGCGCACGCCGCCCTTGACTCACGGGGATTT
CCAAGTCTCCACCCCATTGACGTC
or
(SEQ ID NO: 15)
ATCCCCCGCTCCTCCCCCGCTCCTCCCCCCGCTCCTCCCCCCGCT
CCTCCCCCCGCTCCTCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCCGCGGCCCCGCCCCCAACGCCCGCTC
CTCCCCCCGCTCCCGCGGCCCCGCCCCCAACGCCCGCCGCGCGCG
CGCACGCCGCCCGGACCGCCGCCCGCCTTTTTTGCGCGCCGCCCC
GCCCGCGGGGGGCCCGGGCTGCGCCGCCGCGCTTTAAAGGGCCGC
GCGCGACCCCCGGGGGGTGTGTTTCGGGGGGGGCCCGTTTCTCCC
GCTCCTCCCCCCGCTCCTCCCCCCGCTCCTCCCCCCGCTCCTCCC
CCCGCTCCTCCCCCCGCTCCTCCCCCCGCTCCTCCCCCCGCTCCT
CCCCCCGCTCCTCCCCCCGCTCCTCCCCCCGCTCCTCCCCCCGCT
CCCGCGGCCCCGCCCCCAACGCCCGCTCCTCCCCCCGCTCCCGCG
GCCCCGCCCCCAACGCCCGCCGCGCGCGCGCACGCCGCCCT.

These enhancers can result in higher levels of expression of a linked transgene as compared to an intact HCMV IE expression cassette. They may also act as repressors in some cell types (see, e.g., Kothari S et al, Nucleic Acids Res, 19: 1767, '91; Liu R et al, Nucleic Acids Res 22: 2453, '94; Zweidler-Mckay P A, Mol Cell Biol 16: 4043, '96; Sun B et al, J Cell Biochem 83: 563, '01). The enhancers and expression constructs described herein can be used in delivery and expression of transgenes in cells and tissues both in vitro and in vivo.

Expression constructs can include plasmids, cosmids, viral vectors, or integrated sequences, in which the enhancers are inserted upstream of a promoter so as to increase expression of a transgene linked to the promoter. Viral vectors for use in the present methods and compositions can include recombinant retroviruses, adenovirus, adeno-associated virus, alphavirus, herpesviruses, oncoviruses, and lentivirus, comprising the enhancers described herein inserted upstream of a promoter and optionally a transgene operably linked to the promoter and enhancer for expression in a target tissue.

An exemplary viral vector system useful as an expression construct in the present compositions and methods is the adeno-associated virus (AAV). AAV is a tiny non-enveloped virus having a 25 nm capsid. No disease is known or has been shown to be associated with the wild-type virus. AAV has a single-stranded DNA (ssDNA) genome. AAV has been shown to exhibit long-term episomal transgene expression, and AAV has demonstrated excellent transgene expression in the brain, particularly in neurons. Space for exogenous DNA in AAV is generally limited to an amount of nucleic acid that can physically fit inside the particle. For example, AAV types 1-5 can package up to 6 kb DNA, and in some reports AAV5 has been shown to package up to 8.9 kb DNA. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. Such AAV's can be used for delivery of a nucleic acid comprising an enhancer as described herein. In some embodiments, an AAV suitable for use with an enhancer described herein is AAV1, AAV2, AAV3, AAV4, AAV5, AAV6, AV6.2, AAV7, AAV8, rh.8, AAV9, rh.10, rh.39, rh.43 or CSp3.

The present methods and compositions can include expression constructs comprising oncolytic herpesviruses (oHV), which can be derived from several different types of herpesviruses. The Herpesviridae are a large family of DNA viruses that cause diseases in humans and animals. Herpesviruses all share a common structure and are composed of relatively large double-stranded, linear DNA genomes encoding 80-200 genes encased within an icosahedral protein cage called the capsid, which is itself covered with a tegument layer, wrapped in a lipid bilayer membrane called the envelope. This particle is known as the virion. The large genome provides many non-essential sites for introducing one or more transgenes without inactivating the virus (e.g., without completely inhibiting infection or replication). However, it should be appreciated that oncovirus vectors for use in the present methods and compositions are preferably modified (e.g., replication conditional, attenuated) so that they do not have undesirable effects (e.g., kill normal cells, cause disease). See, e.g., PCT/US2009/002735.

As used herein, oncolytic Herpes virus (oHV) refers to any one of a number of therapeutic viruses having a Herpes virus origin that are useful for killing cancer cells, particularly cancer stem cells, and/or inhibiting the growth of a tumor, for example by killing cancer stem cells of a tumor. Typically, an oncolytic herpesvirus is a mutant version of a wild-type herpesvirus (see, e.g., Table 1 of PCT/US2009/002735). In some cases, the wild-type Herpes virus is of the subfamily alpha (i.e., is a herpes simplex virus type 1 or 2), and this oHV can be referred to as an oncolytic herpes simplex virus (oHSV). In some cases, the oHV is a replication-conditional herpes virus or oHSV. Replication-conditional/selective Herpes viruses are designed to preferentially replicate in actively dividing cells, such as cancer cells, in particular cancer stem cells. Thus, these replication-conditional viruses target cancer cells for oncolysis, and replicate in these cells so that the virus can spread to other cancer cells. In preferred embodiments, replication-conditional Herpes viruses target cancer stem cells for oncolysis, and replicate in these cells so that the virus can spread to other cancer stem cells.

The herpes virus expression constructs can comprise any one of a number of mutations that affect expression of a viral gene. As used herein, the term “gene” encompasses both the regions coding the gene product as well as regulatory regions for that gene, such as a promoter or enhancer, unless otherwise indicated. In most cases, a mutation is in a virulence gene that contributes to the pathogenicity of the virus to a host organism. The mutation may be a point mutation, a deletion, an inversion, or an insertion. Typically, the mutation is an inactivating mutation. As used herein, the term “inactivating mutation” is intended to broadly indicate a mutation or alteration to a gene wherein the expression of that gene is significantly decreased, or wherein the gene product is rendered nonfunctional, or its ability to function is significantly decreased.

Among the viruses described herein are oncolytic herpes simplex viruses (oHSVs) that are genetically altered to be safe and non-pathogenic when administered in a subject, including the nervous system, yet selectively replicate in and kill tumor cells, particularly glioblastoma stem-like cells (GSCs) and other cancer stem cells (see, e.g., U.S. Pat. No. 8,703,120). Several types of oHSV mutants have been developed and are useful in aspects of the methods disclosed herein. For example, one aspect involves viral mutants with defects in the function of a viral gene needed for nucleic acid metabolism, such as thymidine kinase (Martuza, R. L., et al., Science 252:854-856 (1991)), ribonucleotide reductase (RR) (Goldstein, D. J. & Weller, S. K., J. Virol. 62:196-205 (1988); Boviatsis, E. J., et al., Gene Ther. 1:323-331 (1994); Boviatsis, E. J., et al., Cancer Res. 54:5745-5751 (1994); Mineta, T., et al., Cancer Res. 54:3363-3366 (1994)), or uracil-N-glycosylase (Pyles, R. B. and Thompson, R. I., J. Virol. 68:4963-4972 (1994)). Another aspect involves viral mutants with defects in the function of the 734.5 or RL1 gene (Markert J M et al, Neurosurgery 32: 597 (1993); Randazzo B P et al, Virology 211: 94 (1995); Chambers, R., et al., Proc. Natl. Acad. Sci. USA 92:1411-1415 (1995)), which functions as a virulence factor by markedly enhancing the viral burst size of infected cells through suppression of the shutoff of host protein synthesis (Chou, J., et al., Science 250:1262-1266 (1990); Chou, J. and Roizman, B., Proc. Natl. Acad. Sci. USA 89:3266-3270 (1992)). inhibition of autophagy and innate anti-viral immune responses (Kanai R et al, J Virol 86: 4420 (2012); Verpooten D et al, J Biol Chem 284: 1097 (2009)). Other examples include G207 (Mineta, T., et al., Nat. Med 1:938-943 (1995); U.S. Pat. No. 5,585,096), and MGH1 (Kramm, C. M., et al., Hum. Gene Ther. 8:2057-2068 (1997), which possess deletions of both copies of γ-34.5 and an insertional mutation of RR. Additional oHSVs include MG18L with a deletion of the Us3 gene (Kanai et al., Clin Cancer Res. 2011 Jun. 1; 17(11):3686-96); Δ68H-6 with a deletion of the beclin-binding domain of γ-34.5 (Kanai et al., J Virol. 2012 April; 86(8): 4420-4431), and G47Δ with deletion of ICP47 in G207 (WO2002076216; see also Todo et al., Proc Natl Acad Sci USA. 2001 May 22; 98(11): 6396-6401).

A specific example of such a virus is G47Δ (U.S. Pat. Nos. 7,749,745; 8,470,577; WO2002076216), containing; (i) deletions of both copies of γ34.5, the major neurovirulence gene, as well as inhibiting PKR-mediated host protein shutoff, Beclin1-mediated autophagy, and innate anti-viral responses; (ii) deletion of ICP47 and the Us11 promoter, which enhances MHC 1 antigen presentation and complements loss of host protein shutoff, respectively; and (iii) inactivating insertion in ICP6, encoding the large subunit of ribonucleotide reductase that enables replication in non-dividing cells and promoting pathogenesis [5]. The phenotypes of these mutations can be reproduced using HSV with other genetic alterations, such as downregulation, tumor-selective expression, and/or specific amino acid mutations of γ34.5, ICP47, and/or ICP6, and/or upregulation or tumor-selective expression of Us11.

The modified enhancers described herein can be used in combination with a number of promoters. Appropriate regulatory elements can be selected by those of ordinary skill in the art based on, for example, the desired tissue-specificity and level of expression. For example, a cell-type specific or tumor-specific promoter can be used to limit expression of a gene product to a specific cell type (U.S. Pat. No. 5,728,379). This is particularly useful, for example, when a cytotoxic, immunomodulatory, or tumor antigenic gene product is being produced in a tumor cell in order to facilitate its destruction or for a reporter gene (e.g., a fluorescent or detectable protein such as LacZ or GFP) to uniquely identify the oncolytic virus or virus-infected cells. In addition to using tissue-specific promoters, local administration of the viruses described herein can result in localized expression and effect.

Examples of non-tissue specific promoters that can be used in the expression constructs described herein include the early Cytomegalovirus (CMV) promoter (U.S. Pat. No. 4,168,062); the Rous Sarcoma Virus promoter (Norton et al., Molec. Cell. Biol. 5: 281, 1985); DNA virus promoters; adenovirus major late promoter; HSV minimal TK promoter; or cellular (e.g., chicken b-actin or human EF1), or synthetic promoters. Also, HSV promoters, such as HSV-1 IE and IE 4/5 promoters, can be used.

In some embodiments, a combination of a modified enhancer described herein and the HSV IE promoter is used, comprising:

(SEQ ID NO: 16)
CTCCCCCCGCTCCTCCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCTCCCCCCCCCGCTCCCGCGGCCCCGC
CCCCAACGCCCGCTCCTCCCCCCGCTCCCGCGGCCCCGCCCCCAA
CGCCCGCCGCGCGCGCGCACGCCGCCCTTGACTCACGGGGATTTC
CAAGTCTCCACCCCATTGACGTCAATGGGAGTTTGTTTTGGCACC
AAAATCAACGGGACTTTCCAAAATGTCGTAACAACTCCGCCCCAT
TGACGCAAATGGGCGGTAGGCGTGTACGGTGGGAGGTCTATATAA
GCAGAGCTCTCTGGCTAACTAGAGAACCCACTGCTTACTGGCTTA
TCGAAATTAATACGACTCACTATAGGGAGACCCAAGCTTACCGGC
GAAGGAGGGCCACC.

Examples of tissue-specific promoters that can be used in expression constructs described herein include, for example, the prostate-specific antigen (PSA) promoter, which is specific for cells of the prostate; the desmin promoter, which is specific for muscle cells (Li et al., Gene 78: 243, 1989; Li et al., J. Biol. Chem. 266: 6562, 1991; Li et al., J. Biol. Chem. 268: 10403, 1993); the enolase promoter, which is specific for neurons (Forss-Petter et al., J. Neuroscience Res. 16 (1): 141-156, 1986); the beta-globin promoter, which is specific for erythroid cells (Townes et al., EMBO J. 4: 1715, 1985); the tau-globin promoter, which is also specific for erythroid cells (Brinster et al., Nature 283: 499, 1980); the growth hormone promoter, which is specific for pituitary cells (Behringer et al., Genes Dev. 2: 453, 1988); the insulin promoter, which is specific for pancreatic beta cells (Selden et al., Nature 321: 545, 1986); the glial fibrillary acidic protein promoter, which is specific for astrocytes (Brenner et al., J. Neurosci. 14: 1030, 1994); the tyrosine hydroxylase promoter, which is specific for catecholaminergic neurons (Kim et al., J. Biol. Chem. 268: 15689, 1993); the amyloid precursor protein promoter, which is specific for neurons (Salbaum et al., EMBO J. 7: 2807, 1988); the dopamine beta-hydroxylase promoter, which is specific for noradrenergic and adrenergic neurons (Hoyle et al., J. Neurosci. 14: 2455, 1994); the tryptophan hydroxylase promoter, which is specific for serotonin/pineal gland cells (Boularand et al., J. Biol. Chem. 270: 3757, 1995); the choline acetyltransferase promoter, which is specific for cholinergic neurons (Hersh et al., J. Neurochem. 61: 306, 1993); the aromatic L-amino acid decarboxylase (AADC) promoter, which is specific for catecholaminergic/5-HT/D-type cells (Thai et al., Mol. Brain Res. 17: 227, 1993); the proenkephalin promoter, which is specific for neuronal/spermatogenic epididymal cells (Borsook et al., Mol. Endocrinol. 6: 1502, 1992); the reg (pancreatic stone protein) promoter, which is specific for colon and rectal tumors, and pancreas and kidney cells (Watanabe et al., J. Biol. Chem. 265: 7432, 1990); and the parathyroid hormone-related peptide (PTHrP) promoter, which is specific for liver and cecum tumors, and neurilemoma, kidney, pancreas, and adrenal cells (Campos et al., Mol. Endocrinol. 6: 1642, 1992).

Examples of promoters that function specifically in tumor cells include the stromelysin 3 promoter, which is specific for breast cancer cells (Basset et al., Nature 348: 699, 1990); the surfactant protein A promoter, which is specific for non-small cell lung cancer cells (Smith et al., Hum. Gene Ther. 5: 29-35, 1994); the secretory leukoprotease inhibitor (SLPI) promoter, which is specific for SLPI-expressing carcinomas (Garver et al., Gene Ther. 1: 46-50, 1994); the tyrosinase promoter, which is specific for melanoma cells (Vile et al., Gene Therapy 1: 307, 1994; WO 94/16557); the stress inducible grp78/BiP promoter, which is specific for fibrosarcoma/tumorigenic cells (Gazit et al., Cancer Res. 55 (8): 1660, 1995); the AP2 adipose enhancer, which is specific for adipocytes (Graves, J. Cell. Biochem. 49: 219, 1992); the a-1 antitrypsin transthyretin promoter, which is specific for hepatocytes (Grayson et al., Science 239: 786, 1988); the interleukin-10 promoter, which is specific for glioblastoma multiform cells (Nitta et al., Brain Res. 649: 122, 1994); the c-erbB-2 promoter, which is specific for pancreatic, breast, gastric, ovarian, and non-small cell lung cells (Harris et al., Gene Ther. 1: 170, 1994); the a-B-crystallin/heat shock protein 27 promoter, which is specific for brain tumor cells (Aoyama et al., Int. J. Cancer 55: 760, 1993); the basic fibroblast growth factor promoter, which is specific for glioma and meningioma cells (Shibata et al., Growth Fact. 4: 277, 1991); the epidermal growth factor receptor promoter, which is specific for squamous cell carcinoma, glioma, and breast tumor cells (Ishii et al., Proc. Natl. Acad. Sci. U.S.A. 90: 282, 1993); the mucin-like glycoprotein (DF3, MUC1) promoter, which is specific for breast carcinoma cells (Abe et al., Proc. Natl. Acad. Sci. U.S.A. 90: 282, 1993); the mtsl promoter, which is specific for metastatic tumors (Tulchinsky et al., Proc. Natl. Acad. Sci. U.S.A. 89: 9146, 1992); the NSE promoter, which is specific for small-cell lung cancer cells (Forss-Petter et al., Neuron 5: 187, 1990); the somatostatin receptor promoter, which is specific for small cell lung cancer cells (Bombardieri et al., Eur. J. Cancer 31A: 184, 1995; Koh et al., Int. J. Cancer 60: 843, 1995); the c-erbB-3 and c-erbB-2 promoters, which are specific for breast cancer cells (Quin et al., Histopathology 25: 247, 1994); the c-erbB4 promoter, which is specific for breast and gastric cancer cells (Rajkumar et al., Breast Cancer Res. Trends 29: 3, 1994); the thyroglobulin promoter, which is specific for thyroid carcinoma cells (Mariotti et al., J. Clin. Endocrinol. Meth. 80: 468, 1995); the a-fetoprotein promoter, which is specific for hepatoma cells (Zuibel et al., J. Cell. Phys. 162: 36, 1995); the villin promoter, which is specific for gastric cancer cells (Osborn et al., Virchows Arch. A. Pathol. Anat. Histopathol. 413: 303, 1988); and the albumin promoter, which is specific for hepatoma cells (Huber, Proc. Natl. Acad. Sci. U.S.A. 88: 8099, 1991; Miyatake S et al, J Virol 71: 5142, 1997).

Expression vectors comprising the enhancers described herein can include, and thus be used to express, a heterologous nucleic acid sequence (also referred to herein as a transgene) encoding one or more proteins, e.g., therapeutic products, for example, a cytotoxin, an immunomodulatory protein (i.e., a protein that either enhances or suppresses a host immune response to an antigen), a tumor antigen, an antisense RNA molecule, or a ribozyme, under control of the enhancer/promoter. Examples of immunomodulatory proteins include, e.g., cytokines (e.g., interleukins, for example, any of interleukins 1-15, α, β, or γ-interferons, tumor necrosis factor, granulocyte macrophage colony stimulating factor (GM-CSF), macrophage colony stimulating factor (M-CSF), and granulocyte colony stimulating factor (G-CSF)), chemokines (e.g., neutrophil activating protein (NAP), macrophage chemoattractant and activating factor (MCAF), RANTES, and macrophage inflammatory peptides MTP-1a and MTP-1b), complement components and their receptors, immune system accessory molecules (e.g., B7.1 and B7.2), adhesion molecules (e.g., ICAM-1, 2, and 3), adhesion receptor molecules, and adenosine-degrading enzymes (e.g., adenosine kinase and adenosine deaminase, see Zhulai et al., Biomolecules. 2022 March; 12(3): 418). Examples of tumor antigens that can be produced using the present methods include, e.g., the E6 and E7 antigens of human papillomaviras, EBN-derived proteins (Nan der Bruggen et al, Science 254:1643-1647, 1991), mucins (Livingston et al, Curr. Opin. Immun. 4(5):624-629, 1992), such as MUC1 (Burchell et al., hit. J. Cancer 44:691-696, 1989), melanoma tyrosinase, and MZ2-E (Nan der Bruggen et al., 1991) (see also WO 94/16716 and WO2002076216 for a further description of modification of viruses to include genes encoding tumor antigens or cytokines).

An exemplary human IL-12 expression cassette comprising the modified enhancer and an HCMV IE promoter as described herein can comprise the sequence:

(SEQ ID NO: 17)
CTCCCCCCGCTCCTCCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCTCCCCCCCCCGCTCCCGCGGCCCCGC
CCCCAACGCCCGCTCCTCCCCCCGCTCCCGCGGCCCCGCCCCCAA
CGCCCGCCGCGCGCGCGCACGCCGCCCTTGACTCACGGGGATTTC
CAAGTCTCCACCCCATTGACGTCAATGGGAGTTTGTTTTGGCACC
AAAATCAACGGGACTTTCCAAAATGTCGTAACAACTCCGCCCCAT
TGACGCAAATGGGCGGTAGGCGTGTACGGTGGGAGGTCTATATAA
GCAGAGCTCTCTGGCTAACTAGAGAACCCACTGCTTACTGGCTTA
TCGAAATTAATACGACTCACTATAGGGAGACCCAAGCTTACCGGC
GAAGGAGGGCCACCATGGGTCACCAGCAGTTGGTCATCTCTTGGT
TTTCCCTGGTTTTTCTGGCATCTCCCCTCGTGGCCATATGGGAAC
TGAAGAAAGATGTTTATGTCGTAGAATTGGATTGGTATCCGGATG
CCCCTGGAGAAATGGTGGTCCTCACCTGTGACACCCCTGAAGAAG
ATGGTATCACCTGGACCTTGGACCAGAGCAGTGAGGTCTTAGGCT
CTGGCAAAACCCTGACCATCCAAGTCAAAGAGTTTGGAGATGCTG
GCCAGTACACCTGTCACAAAGGAGGCGAGGTTCTAAGCCATTCGC
TCCTGCTGCTTCACAAAAAGGAAGATGGAATTTGGTCCACTGATA
TTTTAAAGGACCAGAAAGAACCCAAAAATAAGACCTTTCTAAGAT
GCGAGGCCAAGAATTATTCTGGACGTTTCACCTGCTGGTGGCTGA
CGACAATCAGTACTGATTTGACATTCAGTGTCAAAAGCAGCAGAG
GCTCTTCTGACCCCCAAGGGGTGACGTGCGGAGCTGCTACACTCT
CTGCAGAGAGAGTCAGAGGGGACAACAAGGAGTATGAGTACTCAG
TGGAGTGCCAGGAGGACAGTGCCTGCCCAGCTGCTGAGGAGAGTC
TGCCCATTGAGGTCATGGTGGATGCCGTTCACAAGCTCAAGTATG
AAAACTACACCAGCAGCTTCTTCATCAGGGACATCATCAAACCTG
ACCCACCCAAGAACTTGCAGCTGAAGCCATTAAAGAATTCTCGGC
AGGTGGAGGTCAGCTGGGAGTACCCTGACACCTGGAGTACTCCAC
ATTCCTACTTCTCCCTGACATTCTGCGTTCAGGTCCAGGGCAAGA
GCAAGAGAGAAAAGAAAGATAGAGTCTTCACGGACAAGACCTCAG
CCACGGTCATCTGCCGCAAAAATGCCAGCATTAGCGTGCGGGCCC
AGGACCGCTACTATAGCTCATCTTGGAGCGAATGGGCATCTGTGC
CCTGCAGTGTTCCTGGAGTAGGGGTACCTGGGGTGGGCGCCAGAA
ACCTCCCCGTGGCCACTCCAGACCCAGGAATGTTCCCATGCCTTC
ACCACTCCCAAAACCTGCTGAGGGCCGTCAGCAACATGCTCCAGA
AGGCCAGACAAACTCTAGAATTTTACCCTTGCACTTCTGAAGAGA
TTGATCATGAAGATATCACAAAAGATAAAACCAGCACAGTGGAGG
CCTGTTTACCATTGGAATTAACCAAGAATGAGAGTTGCCTAAATT
CCAGAGAGACCTCTTTCATAACTAATGGGAGTTGCCTGGCCTCCA
GAAAGACCTCTTTTATGATGGCCCTGTGCCTTAGTAGTATTTATG
AAGACTTGAAGATGTACCAGGTGGAGTTCAAGACCATGAATGCAA
AGCTGCTGATGGATCCTAAGAGGCAGATCTTTCTAGATCAAAACA
TGCTGGCAGTTATTGATGAGCTGATGCAGGCCCTGAATTTCAACA
GTGAGACTGTGCCACAAAAATCCTCCCTTGAAGAACCGGATTTTT
ATAAAACTAAAATCAAGCTCTGCATACTTCTTCATGCTTTCAGAA
TTCGGGCAGTGACTATTGATAGAGTGATGAGCTATCTGAATGCTT
CCTAA.

In some embodiments, an expression construct as described herein, e.g., an oHSV, comprises SEQ ID NO:1 or a sequence at least 80%, 90%, 95%, or 99% identical to nucleotides 93-2490 of SEQ ID NO:1, or to nucleotides 416-2490 of SEQ ID NO:1. The therapeutic product can also be an RNA molecule, such as an antisense RNA molecule that, by hybridization interactions, can be used to block expression of a cellular or pathogen mRNA. Alternatively, the RNA molecule can be a ribozyme (e.g., a hammerhead or a hairpin-based ribozyme) designed either to repair a defective cellular RNA, or to destroy an undesired cellular or pathogen-encoded RNA (see, e.g., Sullenger, Chem. Biol. 2(5):249-253, 1995; Czubayko et al., Gene Ther. 4(9):943-949, 1997; Rossi, Ciba Found. Symp. 209:195-204, 1997; James et al, Blood 91(2):371-382, 1998; Sullenger, Cytokines Mol. Ther. 2(3):201-205, 1996; Hampel, Prog. Nucleic Acid Res. Mol. Bio. 58:1-39, 1998; Curcio et al., Pharmacol. Ther. 74(3):317-332, 1997).

### Methods of Treatment

The expression constructs described herein are useful, e.g., in therapeutic methods, such as, for example, in gene therapy and the treatment of cancer. Oncolytic viruses are particularly well suited for this purpose because they replicate in, and thus destroy, cancer cells but they do not replicate substantially in normal cells, and thus are avirulent. The viruses described herein can also be used in immunization methods, for the treatment or prevention of, for example, infectious diseases, cancer, or autoimmune diseases. An advantageous feature of oncolytic viruses described herein is that, in addition to directly causing lysis of tumor cells, they induce a systemic immune response against tumors. The effects of the viruses described herein are augmented by inserting a heterologous nucleic acid sequence (transgene) encoding a therapeutic product, for example, the cytokine IL-12, under the control of an enhancer sequence described herein in conjunction with a promoter.

Oncolytic Viruses (OVs), e.g., as described above, were initially designed to specifically kill cancer cells, inhibiting tumor growth by direct oncolytic activity. Thus, oHSV therapy may be performed using an oncolytic virus (such as G47Δ) expressing IL-12 under control of an altered enhancer, e.g., a G47ΔhIL12A oHSV. G47Δ has been shown to be safe and efficacious after intratumoral administration in glioblastoma patients and is conditionally approved for the treatment of recurrent glioma in Japan [9]. Loss of γ34.5, such as in oHSV G207 [4], 1716 [18], or M032 [19] that are in clinical trial, leads to a block in virus replication in GSCs [7, 20]. Cancer stem cells are a subset of tumor cells that drive and sustain tumor growth and manipulate the tumor microenvironment to resist many therapeutic agents, and thus represent an important and possibly essential target for therapy [21]. Glioblastoma, the most malignant primary brain tumor in adults, is one of the tumors where cancer stem cells have been isolated and characterized [15]. The viruses described herein, e.g., comprising IL12 under the control of an altered enhancer, e.g., G47ΔhIL12A oHSV, can be used to treat a solid cancer in a subject as these viruses replicate in, and thus destroy dividing cells, such as cancer cells, but are avirulent to other cells. Examples of cancers that can be treated include solid tumors, e.g., carcinoma or sarcoma. In some embodiments, the cancer is a nervous-system tumor, for example, astrocytoma, oligodendroglioma, meningioma, neurofibroma, glioma, glioblastoma, ependymoma, schwannoma, neurofibrosarcoma, neuroblastoma, pituitary tumor (e.g., pituitary adenoma), and medulloblastoma cells. Other types of tumor cells that can be killed, pursuant to the present invention, include, for example, skin cancer (e.g., melanoma), prostate carcinoma, renal cell carcinoma, pancreatic cancer, breast cancer (e.g., triple negative breast cancer), lung cancer, colon cancer, gastric cancer, fibrosarcoma, squamous cell carcinoma, neuroectodermal, thyroid tumor, lymphoma, hepatoma, mesothelioma, and epidermoid carcinoma cells, as well as other cancer cells mentioned herein. Also as is noted above, the viruses described herein, which induce a systemic immune response to cancer, can be used to prevent or treat cancer metastasis.

As is noted above, the viruses described herein can be used in in vivo methods, for example, to kill a cancer cell (e.g., using an oncolytic virus) and/or to introduce a therapeutic gene product into the cell (e.g., for gene therapy). To carry out these methods, the viruses described herein can be administered by any conventional route used in medicine. For example, a virus as described herein can be administered directly into a tissue in which an effect, e.g., cell killing and/or therapeutic gene expression, is desired, for example, by direct injection or by surgical methods (e.g., stereotactic injection into a brain tumor; Pellegrino et al., Methods in Psychobiology (Academic Press, New York, New York, 67-90, 1971)). An additional method that can be used to administer vectors into the brain is the convection method described by Bobo et al. (Proc. Natl. Acad. Sci. U.S.A. 91:2076-2080, 1994) and Morrison et al. (Am. J. Physiol. 266:292-305, 1994). In the case of tumor treatment, as an alternative to direct tumor (intratumoral) injection, surgery can be carried out to remove the tumor, and the vectors as described herein can be inoculated into the resected tumor bed to ensure destruction of any remaining tumor cells. Alternatively, the vectors can be administered via a parenteral route, e.g., by an intravenous, intraarterial, intracerebroventricular, intrathecal, subcutaneous, intraperitoneal, intradermal, intraepidermal, or intramuscular route, or via a mucosal surface, e.g., an ocular, intranasal, pulmonary, oral, intestinal, rectal, vaginal, or urinary tract surface.

A number of formulations are known for introducing viruses into cells in mammals, such as humans, can be used in the methods and compositions described herein. (See, e.g., Remington's Pharmaceutical Sciences (18th edition), ed. A. Gennaro, 1990, Mack Publishing Co., Easton, PA). However, the viruses can be simply diluted in a physiologically acceptable solution, such as sterile saline or sterile buffered saline, with or without an adjuvant or carrier.

The amount of virus to be administered depends, e.g., on the specific goal to be achieved, the strength of any promoter used in the virus, the condition of the mammal (e.g., human) intended for administration (e.g., the weight, age, and general health of the mammal), the mode of administration, and the type of formulation. In general, a therapeutically or prophylactically effective dose of, e.g., from about 10¹ to 10¹⁰ plaque-forming units (pfu), for example, from about 5×10⁶ to 1×10⁹ pfu, e.g., although the most effective ranges may vary from host to host, as can readily be determined by one of skill in this art. Also, the administration can be achieved in a single dose or repeated at intervals, as determined to be appropriate by those of skill in this art.

See, e.g., Apolonio et al., World J Virol. 2021 Sep. 25; 10(5): 229-255.

Combinations with Immunotherapies: Immune Checkpoint Inhibitors and Inhibitors of the Adenosine Pathway

The viruses described herein can synergize with other immunomodulatory pharmaceutical agents. In previous work, an oHSV expressing mouse IL-12 from the ICP6 locus of G47Δ (G47ΔmIL12) was used to examine the effects of IL-12 expression in vivo, as hIL-12 is not active in mice. G47ΔmIL12 extended survival, but didn't cure C57BL/6 mice bearing non-immunogenic intracerebral mouse GSC-derived tumors [17]. Immune checkpoint inhibitors (ICI) have not demonstrated overall survival improvements in glioblastoma patients in randomized phase 2 or 3 clinical trials, either alone or in combination (anti-PD-1 and anti-CTLA-4) [46]. Combination of G47ΔmIL12 with single ICIs further extended survival modestly but doesn't reproducibly result in a complete response in mice [11]. However, combining two immune checkpoint inhibitors (anti-PD-1 and anti-CTLA-4) with G47ΔmIL12 elicited a complete remission in most treated mice bearing intracerebral mouse GSC-derived tumors [11]. Importantly, the mice with a complete response were protected from tumor rechallenge with a 5-fold increased number of the same GSCs implanted in the contralateral hemisphere, demonstrating immunological memory, whereas all naive age-matched mice succumbed to tumor growth [11].

Thus, these viruses can be used not only to treat a given tumor, to which they may be directly administered, but also to treat invasive nervous system tumors and prevent or treat cancer metastasis.

It has become clear that a major component of anti-tumor efficacy is due to the induction of systemic immune responses against the tumor in which OV is growing [47, 48]. This has been termed immunovirotherapy via in-situ vaccination [49, 50, 51] and can be improved by having the virus express a favorable immunomodulatory molecule [52, 53]. Thus, an OV can be used not only to treat infected cancer cells, but also uninfected normal cells or cancer cells at a distance in invasive nervous system tumors and cancer metastasis. In a stringent immunocompetent GSC intracranial mouse model, G47Δ expressing mouse IL-12 used in combination with two immune checkpoint inhibitors (ICIs) favorably altered the tumor microenvironment (TME) and resulted in a complete response in the majority of mice [11]. The mechanism involves in part polarizing tumor macrophages from M2 to anti-tumor M1 phenotype and depends on CD4+ T cells [11]. While oHSVs lacking therapeutic transgenes induce anti-tumor immune responses, these are typically insufficient to overcome the immune-suppressive TME and systemic immunosuppression occurring in many cancers, especially GBM [12]. Most effective cancer therapy requires multiple therapeutic agents. Thus, oHSV therapy may be performed using multiple therapeutic modes, e.g., as disclosed here with G47Δ expressing IL-12 under control of an altered enhancer, e.g., in combination with multiple ICIs.

The present methods can include administering one or more immunotherapy agents, e.g., immune checkpoint inhibitors, e.g., an inhibitor of PD-1 signaling, e.g., an antibody that binds to PD-1, CD40, or PD-L1, or an inhibitor of Tim3 or Lag3, e.g., an antibody that binds to Tim3 or Lag3, or an antibody that binds to CTLA-4, or an antibody that binds to T-cell immunoglobulin and ITIM domains (TIGIT). In some embodiments, an anti-PD-1 and anti-CTLA-4 are used together.

Exemplary anti-PD-1 antibodies that can be used in the methods described herein include those that bind to human PD-1; an exemplary PD-1 protein sequence is provided at NCBI Accession No. NP_005009.2. Exemplary antibodies are described in U.S. Pat. Nos. 8,008,449; 9,073,994; and US20110271358, including PF-06801591, AMP-224, BGB-A317, BI 754091, JS001, MEDI0680, PDR001, REGN2810, SHR-1210, TSR-042, pembrolizumab, nivolumab, avelumab, pidilizumab, and atezolizumab.

Exemplary anti-CD40 antibodies that can be used in the methods described herein include those that bind to human CD40; exemplary CD40 protein precursor sequences are provided at NCBI Accession No. NP_001241.1, NP_690593.1, NP_001309351.1, NP_001309350.1 and NP_001289682.1. Exemplary antibodies include those described in WO2002/088186; WO2007/124299; WO2011/123489; WO2012/149356; WO2012/111762; WO2014/070934; US20130011405; US20070148163; US20040120948; US20030165499; and U.S. Pat. No. 8,591,900, including dacetuzumab, lucatumumab, bleselumab, teneliximab, ADC-1013, CP-870,893, Chi Lob 7/4, HCD122, SGN-4, SEA-CD40, BMS-986004, and APX005M. In some embodiments, the anti-CD40 antibody is a CD40 agonist, and not a CD40 antagonist.

Exemplary CTLA-4 antibodies that can be used in the methods described herein include those that bind to human CTLA-4; exemplary CTLA-4 protein sequences are provided at NCBI Acc No. NP_005205.2. Exemplary antibodies include those described in Tarhini and Iqbal, Onco Targets Ther. 3:15-25 (2010); Storz, MAbs. 2016 January; 8(1):10-26; US2009025274; U.S. Pat. Nos. 7,605,238; 6,984,720; EP1212422; U.S. Pat. Nos. 5,811,097; 5,855,887; 6,051,227; 6,682,736; EP1141028; and U.S. Pat. No. 7,741,345; and include ipilimumab, Tremelimumab, and EPR1476.

Exemplary anti-PD-L1 antibodies that can be used in the methods described herein include those that bind to human PD-Li; exemplary PD-L1 protein sequences are provided at NCBI Accession No. NP_001254635.1, NP_001300958.1, and NP_054862.1. Exemplary antibodies are described in US20170058033; WO2016/061142A1; WO2016/007235A1; WO2014/195852A1; and WO2013/079174A1, including BMS-936559 (MDX-1105), FAZ053, KN035, Atezolizumab (Tecentriq, MPDL3280A), Avelumab (Bavencio), and Durvalumab (Imfinzi, MEDI-4736).

Exemplary anti-Tim3 (also known as hepatitis A virus cellular receptor 2 or HAVCR2) antibodies that can be used in the methods described herein include those that bind to human Tim3; exemplary Tim3 sequences are provided at NCBI Accession No. NP_116171.3. Exemplary antibodies are described in WO2016071448; U.S. Pat. No. 8,552,156; and US PGPub. Nos. 20180298097; 20180251549; 20180230431; 20180072804; 20180016336; 20170313783; 20170114135; 20160257758; 20160257749; 20150086574; and 20130022623, and include LY3321367, DCB-8, MBG453 and TSR-022.

Exemplary anti-Lag3 antibodies that can be used in the methods described herein include those that bind to human Lag3; exemplary Lag3 sequences are provided at NCBI Accession No. NP_002277.4. Exemplary antibodies are described in Andrews et al., Immunol Rev. 2017 March; 276(1):80-96; Antoni et al., Am Soc Clin Oncol Educ Book. 2016; 35:e450-8; US PGPub. Nos. 20180326054; 20180251767; 20180230431; 20170334995; 20170290914; 20170101472; 20170022273; 20160303124, and include BMS-986016.

Exemplary anti-TIGIT antibodies that can be used in the methods described herein include those that bind to human TIGIT; an exemplary human TIGIT sequence is provided at NCBI Accession No. NP_776160.2. Exemplary antibodies include AB154; MK-7684; BMS-986207; ASP8374; Tiragolumab (MTIG7192A; RG6058); (Etigilimab (OMP-313M32)); 313R12. See, e.g., Harjunpaa and Guillerey, Clin Exp Immunol 2019 Dec. 11[Online ahead of print], DOI: 10.1111/cei.13407; 20200062859; and 20200040082.

Alternatively or in addition, inhibitors of the adenosine pathway, which is immunosuppressive in the tumor microenvironment, can also be administered. Such inhibitors include small molecule inhibitors and antibodies to CD39, CD73, A2a Receptor (A2aR), and A2bR. See e.g., Zahavi and Hodge, Int J Mol Sci 2023 24: 8871. Anti-CD39 antibodies can include SRF617, ES014, PUR001, TTX-030, JS019, ES002023, INCA00186, CPI-006, and IPH5201. Small molecule inhibitors of CD73 include AB680 and ORIC-533. Anti-CD73 mAbs include PT199, Oleclumab, TJ004309, AK119, CPI-006, JAB-BX102, AGEN1423, IBI325, IPH5301, Sym024, and NZV930. Small-molecule A2A Receptor inhibitors include TT-10, inupadenant, and AZD4635. Small-Molecule A2B Receptor Inhibitors include TT-4 and PBF-1129. Small-molecule dual A2A/A2B receptor inhibitors include M1069, INCB106385, and etrumadenant.

### EXEMPLARY SEQUENCES AND CONSTRUCTS

In some embodiments, the sequence of a protein or nucleic acid used in a composition or method described herein is at least 80%, 85%, 90%, 95%, 97%, 98%, or 99% identical to a reference sequence set forth herein. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

### EXAMPLES

The invention is further described in the following examples, which do not limit the scope of the invention described in the claims.

### Materials and Methods

The following materials and methods were used in the Examples below.

### Cells and Viruses

Vero (African Green Monkey kidney cells) cells (ATCC, Manassas, VA) were grown in Dulbecco's modified Eagle's medium (DMEM; Mediatech Corning) supplemented with 10% calf serum (HyClone, Cytiva). Human GBM stem-like cells (GSCs) MGG4, MGG8, MGG23, and MGG123 have been described [15, 16]) and were grown in EF medium composed of Neurobasal medium (Invitrogen, ThermoFisher) supplemented with 3 mmol/L L-Glutamine (Cellgro), 1×B27 supplement (Gibco, ThermoFisher), 0.5×N2 supplement (Gibco), 2 μg/mL heparin (Sigma-Aldrich), 20 ng/mL recombinant human EGF (R&D systems, Bio-techne), 20 ng/mL recombinant human FGF2 (Peprotech), and 0.5×penicillin G/streptomycin sulfate/amphotericin B complex (Cellgro, Corning). NK-92® MI (ATCC CRL-2408) cells were grown in the complete medium as described by vendor. All cells were confirmed to be mycoplasma-free (LookOut mycoplasma kit; Sigma-Aldrich, St. Louis MO). Wild-type HSV-1 strain F was obtained from Dr. B. Roizman (University of Chicago, IL). G47Δ (γ34.5Δ ICP6⁻, ICP47Δ) was derived from G207 [5]. Virus stocks were generated from low-multiplicity infections of Vero cells and purified [2].

### Construction of G47ΔhIL-12 Virus

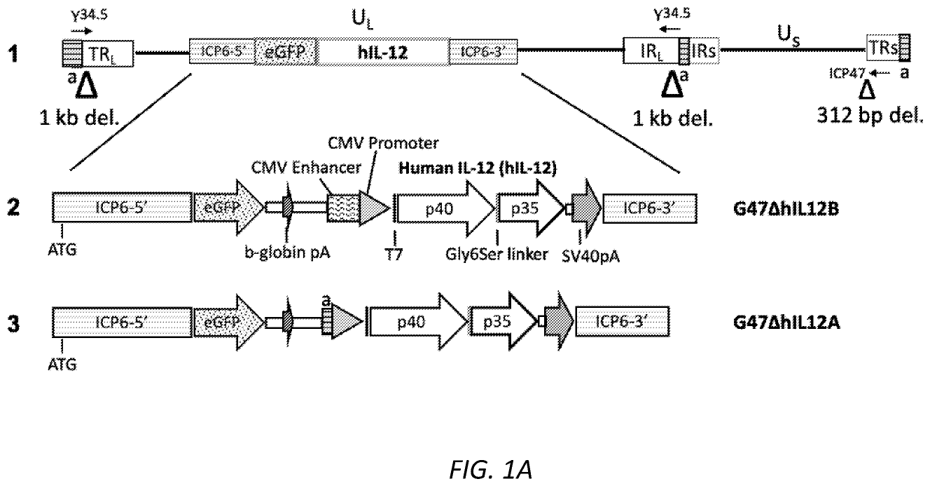

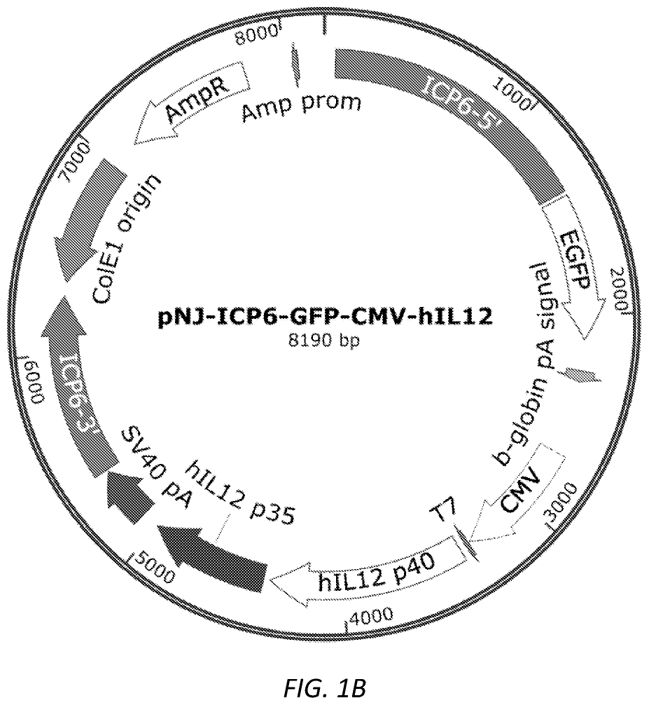

The peptide-linked p40 and p35 subunits of hIL12 were isolated from plasmid pORF-hIL-12g2 (InvivoGen, Cambridge, MA), cloned downstream the HCMV immediate-early (IE) enhancer/promoter (from pcDNA1, Invitrogen), and sub-cloned into ICP6 shuttle plasmid pKX2-βG3 [2](kindly provided by S. Weller, University of Connecticut). Next, the gene encoding enhanced Green Fluorescent Protein (eGFP) from pCAG-Cre:GFP (Addgene, Watertown, MA) was inserted into the ICP6 ORF to express an ICP6-GFP fusion protein under the viral ICP6 promoter. The structure of the resulting shuttle plasmid, pNJ-ICP6-GFP-CMV-hIL-12 (FIG. 1A) was verified by restriction endonuclease digestion and DNA sequencing. The shuttle vector, pNJ-ICP6-GFP-CMV-hIL-12, and G47Δ viral DNA were co-transfected into Vero cells using Lipofectamine 2000 (Invitrogen), followed by several rounds of screening, and isolation of eGFP-expressing recombinant G47Δ-hIL12 virus. Multiple independent isolates were purified by limiting dilution and three rounds of plaque purification.

Selected viral clones were purified in large scale as follows. Vero cells were infected with G47ΔhIL12 clones at a multiplicity of infection (MOI) of 0.05, the cells harvested at 90% cytopathic effect (CPE) and pelleted by centrifugation (1,200 rpm for 10 min). Viral particles were released by three cycles of freezing-thawing followed by sonication with 30% output at 4° C. To degrade cellular DNA, lysates were treated with Benzonase (AIC ultrapure grade; Sigma-Aldrich) at 300 units/ml in the presence of 2 mM MgCl₂ at 37° C. for 30 min. Cellular debris was pelleted by centrifugation at 4° C. and the clarified lysate was filtered through a series of syringe filters, (5.0-, 0.8-, and 0.45-μm; PVDF type from MilliporeSigma, Burlington, MA). The filtered viral lysate was loaded onto a 25% sucrose/HBSS buffer cushion and centrifuged at 11.5K rpm for 90 min at 4° C. in a F13-14×50CY rotor. The pelleted virus was washed carefully with PBS, resuspended in PBS/10% glycerol, and sonicated/rocked until the pellet was dislodged and resuspended. The resuspended virus was aliquoted and stored at −80° C. The virus titer was determined by plaque assay [54].

### Preparation and Analysis of G47ΔhIL12 Viral DNA

G47ΔhIL12 viral DNA was purified as previously described with some modifications [28]. Briefly, Vero cells were infected with virus at an MOI of 1.5 and harvested when total CPE was observed. Cells were pelleted by centrifugation, resuspended in RSB buffer (10 mM Tris-HCl pH8.0, 10 mM KCl, 1.5 mM MgCl2), and incubated for 20 min on ice to let cells swell. NP40 (0.5%) and 1.0 mg/ml RNAseA were added, the mixture centrifuged at 2000 rpm 4° C. for 5 min, and the cytoplasmic supernatant collected. The pellet was resuspended in RSB, treated with 0.25% TritonX-100, centrifuged at 2,000 rpm 4° C. for 5 min, and the supernatant added to the cytoplasmic supernatant. The combined supernatant was treated with 10 mM EDTA, 0.4% SDS, and 250 μg/ml Proteinase K, and incubated at 37° C. overnight. Viral DNA was purified by phenol-chloroform extraction and ethanol precipitation. For restriction analysis, viral DNA was digested with Hind III and Bgl II and separated by electrophoresis in 0.6% agarose/Tris-acetate-EDTA buffer.

### Viral DNA Sequencing

The combination of two sequencing technologies, Next-Generation Sequencing (NGS) and the Pacific Biosciences (PacBio, Menlo Park CA) long read sequencing were used to have a more accurate characterization of the G47ΔhIL12 genome. NGS was performed by the MGH CCIB DNA core facility on an Illumina MiSeq short-read sequencing utilizing V2 chemistry. A preliminary G47Δ reference sequence was generated from the HSV-1 strain F sequence (GenBank accession number GU734771) and edited with the introduced genome alterations according to the literature [2, 5, 29, 55] using Geneious Prime 2022.0.2 (geneious.com). Sequencing reads from G47Δ were aligned to the preliminary reference sequence and the consensus was extracted from this alignment file to produce a validated G47Δ reference sequence where all detected features were manually reviewed and annotated. Both the internal repeat long (IR_{L}) and internal repeat short (IR_{S}) sequences from HSV-1 strain F genomic sequence were defined as reference spots. The unique long (U_{L}) and short (U_{S}) regions were then individually inverted to forward and reverse relative orientations to generate the four possible genomic isomers: U_{L}-forward(fw)/U_{S}-reverse(rv); U_{L}-fw/U_{S}-fw; U_{L}-rv/U_{S}-fw and U_{L}-rv/U_{S}-rv. To produce a preliminary G47ΔhIL12 reference sequence, changes in the ICP6 open reading frame (ORF) due to recombination with pNJ-ICP6-eGFP-CMV-hIL12 were introduced to the validated G47Δ reference sequence. The Geneious “Map to Reference” function was used to align the G47ΔhIL12 NGS results (˜250,000 142 bp reads). To resolve ambiguous results of some questionable sequences obtained from NGS and gain deeper coverage, PacBio long-read sequencing on G47ΔhIL12A was performed at the Broad Institute, Boston using the PacBio Sequel II platform with circular consensus sequencing (CCS). A 10 kb CCS library was generated with sheared DNA (larger HMW fragments of at least 40 kb) using one SMRT cell, and results were aligned using the Geneious “Map to Reference” function. Due to the longer read length, the multiple isoforms of the HSV-1 genome were more problematic during alignment. To address this, the linear reference sequence was circularized and used for subsequent alignment similar to the assembly loop alignment described previously (Jiao et al., Microbiol Resour Announc. 2019 September; 8(39): e00993-19). Because coverage was poor within the ICP6 region, additional analysis sought to clarify the sequence of this region. PacBio sequencing reads were filtered to those that contained a portion of at least one of the following sequences: CMV promoter, hIL-12 coding sequence, or GFP. The remaining 94 reads were de novo assembled to produce 8 contigs, the most robust of which encompassed 54 reads and spanned the region of interest. The NGS reads were then mapped to the consensus of this contig and the results of this alignment were included in the final G47ΔhIL12A sequence.

Virus replication and cytotoxicity assays.

Virus yield assay. Vero cells, 5×10⁴/well, were plated in 24 well plates, incubated overnight at 37° C., infected with the indicated viruses at an MOI of 1, and harvested with supernatants at the indicated time points. Dissociated GSCs were resuspended at 5×10⁶ cells/ml, and infected for 45 min at 37° C. Cells were then plated at 2 or 1×10⁴ cells in 24 or 48-well plates, incubated at 37° C., and supernatants harvested at indicated time points. After freeze-thawing and sonication, the titers of infectious viruses were determined by plaque assay on Vero cells.

Cell viability assay. GSCs were seeded in 96-well plates (5×10³ cells/well), treated 24 hrs later with the indicated MOI of virus, and incubated at 37° C. for 4 days. Cell viability was measured by determining cell metabolic activity using an NADPH-dependent CellTiter96 AQueous One Solution Cell Viability (MTS) Assay kit (Promega, Madison, WI), according to the manufacturer's instructions. Experiments were performed in triplicate and repeated at least 3 times.

Plaque reduction assay. Vero cells in 6 well plates were infected with 300 plaque forming units (PFU) of oHSV in the presence of increasing concentrations of acyclovir (Sigma-Aldrich) and 0.1% human IgG. Cells were fixed 72 hours post-infection (hpi) (G47Δ) or 120 hpi (G47ΔhIL12A), stained, and the plaques counted. Dose-response curves were fitted with a least-squares regression method using GraphPad Prism version 9.00 (GraphPad Software, Inc., San Diego, CA) and IC50 values determined.

hIL12 and hIFNγ ELISA Assays

HIL12 levels were measured with the human p70 Quantikine ELISA kit (R&D Systems, Bio-Techne, Minneapolis, MN). Functional hIL12 was assessed using human NK cell line NK-92MI (ATCC (CRL-2408)) [38] and hIL-12 induced IFN-γ production [39]. Recombinant hIL12 (R-hIL12; R&D Systems) with known functional activity was used as a positive control. NK-92MI cells were seeded in a 48-well plate at 2×10⁴ cells/well. hIL12 produced by virus-infected cells 64 hpi or R-hIL12 was added to NK-92MI cell cultures at the indicated concentrations (with or without 10 μg/ml heparin). HIL12-dependent secretion of IFN-γ into the supernatant was quantified after 24 hrs via ELISA (Human IFN-gamma Quantikine ELISA Kits (R&D systems)).

### In Vivo Safety Assay

HSV-susceptible BALB/c female mice (aged 8 wk), obtained from the NCI Frederick, MD, were stereotactically inoculated (right striatum, 2.2-mm lateral from Bregma and 2.5-mm deep) with 3 μl of virus or PBS. Mice were followed for neurologic symptoms, weight loss, and euthanized when moribund. The neurologic score quantified 3 parameters, general appearance (A), spontaneous activity (S), and response to external stimuli or neurological deficits (R), which were each scored 1 for severely impaired, 2 for moderately impaired, 3 for slightly impaired, and 4 for normal, and the values totaled. All mouse procedures were approved by the Institutional Animal Care and Use Committee at Massachusetts General Hospital.

### Histology

Mice were perfused with 4% paraformaldehyde, brains removed, fixed in 4% paraformaldehyde, and paraffin blocks prepared. Seven μm sections were subjected to hematoxylin (Richard Allen Sci, ThermoFisher) & eosin (Fisher Sci, ThermoFisher) staining and immunohistochemistry with a polyclonal rabbit anti-HSV antibody (B0114; Dako, Agilent, Santa Clara CA).

### Statistical Analysis

Comparisons of data for virus yield and hIL-12 were analyzed using a 2-tailed unpaired t test with Welch's correction. The IC50's were determined from a nonlinear regression fit of the dose-response curves. p values<0.05 were considered statistically significant. Prism 10 software (GraphPad Software, Inc., San Diego, CA) was used for analysis.

### Example 1. Construction and Isolation of G47ΔhIL12

G47ΔhIL12 was constructed by inserting a promoter-driven hIL-12 coding sequence cassette into G47Δ and isolating the resultant G47Δ-hIL12 recombinant virus. A plasmid containing the open reading frame encoding linked p40 and p35 subunits of hIL-12 (pORF-hIL-12 G2) was obtained from InvivoGen. HIL-12 expression is driven by the HCMV IE enhancer/promoter. The hIL-12 expression cassette was sub-cloned into the ICP6 shuttle plasmid pKX2-βG3 [2, 27], where it was flanked by HSV ICP6 sequences homologous to the targeted gene locus within the HSV genome. Next, an enhanced Green Fluorescent Protein (eGFP) coding gene from pCAG-Cre:GFP (Addgene) was cloned into the ICP6 ORF to express an ICP6-GFP fusion protein under the endogenous ICP6 viral promoter. The structure of the resulting plasmid, pNJ-ICP6-eGFP-CMV-hIL12 (FIG. 1), was verified by restriction endonuclease digestion and sequencing.

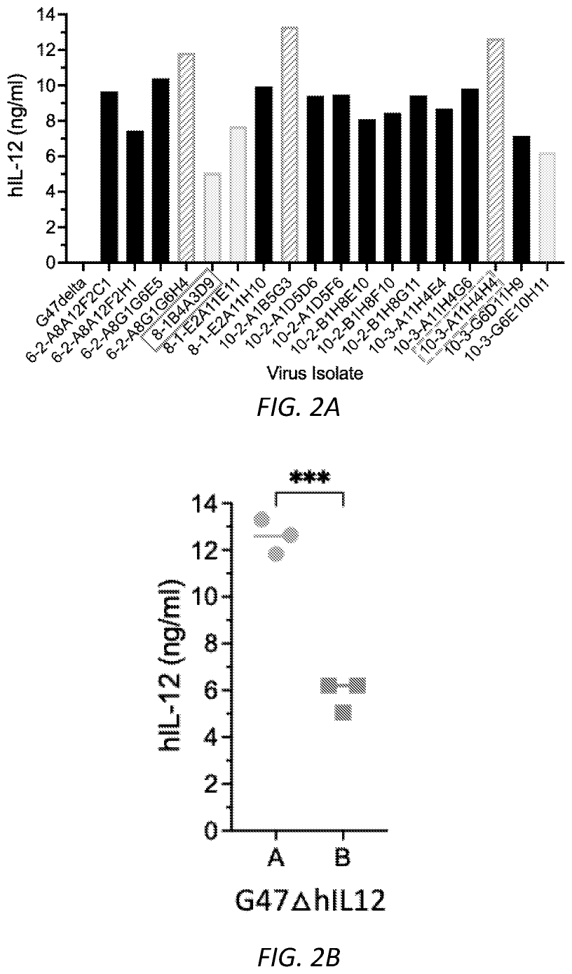

Purified G47Δ DNA and pNJ-ICP6-eGFP-CMV-hIL12 DNA were co-transfected into Vero cells using Lipofectamine 2000. Homologous recombination resulted in the GFP-hIL12 expression cassette being inserted into the ICP6 locus of G47Δ in place of LacZ (FIG. 1A). Recombinant viruses were identified as green plaques under fluorescent microscopy and clear plaques after X-gal staining. Each recombinant virus was subjected to three rounds of plaque purification at limiting dilution, each time confirming the clear plaque phenotype and GFP expression (GFP+/LacZ− plaques), and hIL-12 production. At the 2nd round of purification, 18 virus clones were compared for hIL-12 production (FIG. 2A). Supernatants from individual virus isolates were harvested and examined for hIL-12 production by ELISA. There was a large variability in hIL-12 expression between different isolates (FIG. 2A), which may have reflected differences in the virus sequences. We divided the virus isolates into two groups; those whose infected cell lysates had high and low hIL-12 levels, which we designated G47ΔhIL12A (A for augmented; reference isolate 10-3-A11H4H4 (dashed line box)) and G47ΔhIL12B (reference isolate 8-1B4A3D9 (solid line box). There was an approximately 2-fold significant difference in hIL-12 levels between the top 3 virus isolates in each group (FIG. 2B).

Two clones, each with the highest and lowest hIL-12 expression, were characterized further. The selected viruses were purified after infection of Vero cells at a low multiplicity of infection (MOI). Virus was semi-purified from infected Vero cells, and then viral DNA was purified by phenol-chloroform extraction and ethanol precipitation.

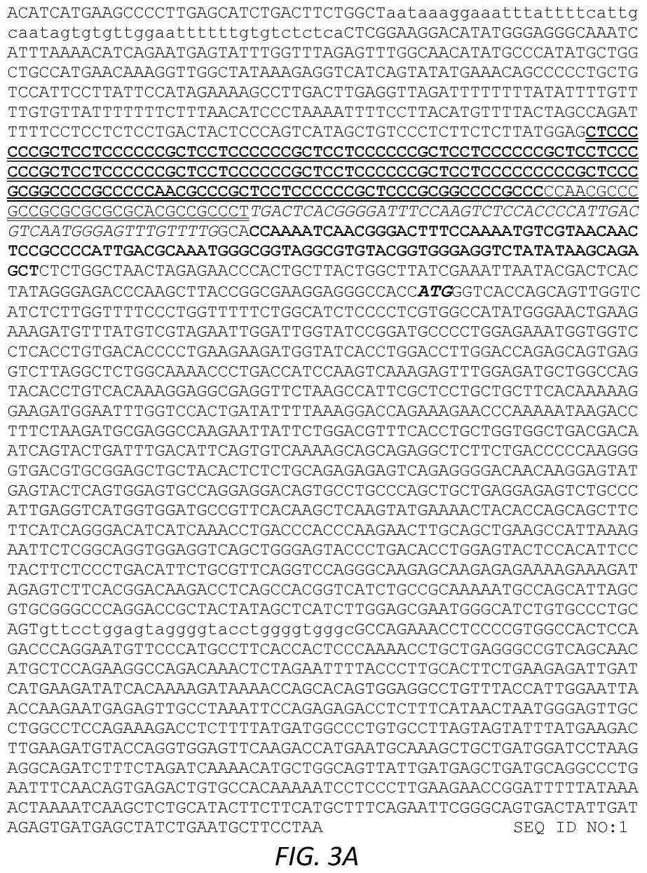

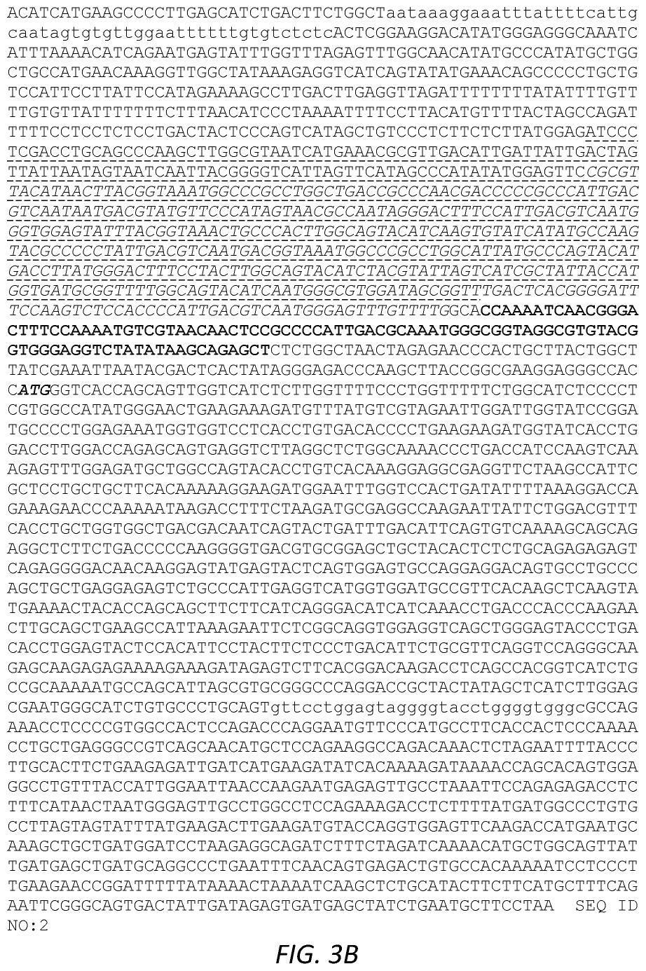

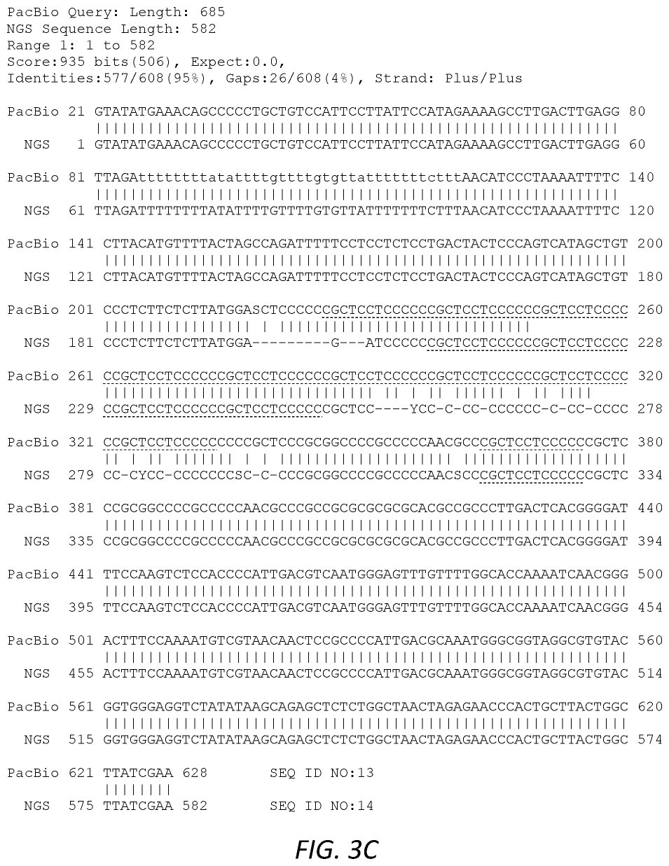

To validate the structure of the recombinant virus isolates, Next-Generation Sequencing (NGS) was performed on an Illumina MiSeq short-read sequencing instrument utilizing V2 chemistry. The alignment of HSV genomes from NGS short reads is complicated due to the high G-C content and repeat sequences, and often requires a reference genome to align the sequence reads. Therefore, a preliminary G47Δ reference sequence was generated from the HSV-1 strain F sequence (GenBank accession number GU734771) and edited with the introduced genome alterations according to the literature [2, 5, 29] using Geneious Prime 2022.0.2 (geneious.com). Sequencing reads from G47Δ were aligned to the preliminary reference sequence, and the consensus was extracted from this alignment file to produce a validated G47Δ reference sequence with all detected features manually reviewed. The unique long (U_{L}) and short (U_{S}) regions were then individually inverted to forward and reverse relative orientations to generate the four possible genomic isomers: U_{L}-forward(fw)/U_{S}-reverse(rv); U_{L}-fw/U_{S}-fw; U_{L}-rv/U_{S}-fw and U_{L}-rv/U_{S}-rv. To produce a preliminary G47ΔhIL12 reference sequence, changes in the ICP6 open reading frame (ORF) due to recombination with pNJ-ICP6-eGFP-CMV-hIL12 were introduced to the validated G47Δ reference sequence. The Geneious “Map to Reference” function was used to align the G47ΔhIL12 NGS results to the preliminary reference sequence described above. This identified two novel genetic alterations arising in G47ΔhIL12A in the region upstream of hIL12: (i) deletion of the HCMV enhancer (465 bps) and (ii) addition/insertion of viral terminal repeat ‘a’ sequences, including 6 DR2 repeat sequence units (CGCTCCTCCCCC (SEQ ID NO:7); J02223.1). This was in contrast to the expected sequences in G47ΔhIL12B (see FIGS. 3A-B).

Because of the difficulty in sequencing and aligning reads due to the high GC content and repeat sequences [30], PacBio long-read sequencing was performed on G47ΔhIL12A using the PacBio Sequel II platform with circular consensus sequencing (CCS). Due to the longer read length, the multiple isoforms of the HSV-1 genome were more problematic during alignment. To address this, the linear reference sequence was circularized and used for subsequent alignment similar to the assembly loop alignment described previously (Jiao et al., Microbiol Resour Announc. 2019 September; 8(39): e00993-19). Because coverage was poor within the ICP6 region, additional analysis sought to clarify the sequence of this region. PacBio sequencing reads were filtered to those that contained a portion of at least one of the following sequences: CMV promoter, hIL-12 coding sequence, or GFP. The remaining 94 reads were de novo assembled to produce 8 contigs, the most robust of which encompassed 54 reads and spanned the region of interest. The NGS reads were then mapped to the consensus of this contig and the results of this alignment were included in the final G47ΔhIL12A sequence.

The PacBio sequence was identical to the NGS sequence in the deletion/insertion region of ICP6 except for an additional 26 bps, creating 10 DR2 repeat sequences (FIG. 3C, [symbol] ). An additional sequence was obtained that contained a 579 bp insert, including a homologous sequence to strain F ‘a’ sequence reiteration set 3 (151939-152093; GenBank GU734772.1) and 24 DR2 repeat sequences.

(SEQ ID NO: 15)
ATCCCCCGCTCCTCCCCCGCTCCTCCCCCCGCTCCTCCCCCCGCT
CCTCCCCCCGCTCCTCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCCGCGGCCCCGCCCCCAACGCCCGCTC
CTCCCCCCGCTCCCGCGGCCCCGCCCCCAACGCCCGCCGCGCGCG
CGCACGCCGCCCGGACCGCCGCCCGCCTTTTTTGCGCGCCGCCCC
GCCCGCGGGGGGCCCGGGCTGCGCCGCCGCGCTTTAAAGGGCCGC
GCGCGACCCCCGGGGGGTGTGTTTCGGGGGGGGCCCGTTTCTCCC
GCTCCTCCCCCCGCTCCTCCCCCCGCTCCTCCCCCCGCTCCTCCC
CCCGCTCCTCCCCCCGCTCCTCCCCCCGCTCCTCCCCCCGCTCCT
CCCCCCGCTCCTCCCCCCGCTCCTCCCCCCGCTCCTCCCCCCGCT
CCCGCGGCCCCGCCCCCAACGCCCGCTCCTCCCCCCGCTCCCGCG
GCCCCGCCCCCAACGCCCGCCGCGCGCGCGCACGCCGCCCT

This may reflect heterogeneity in the size of the terminal repeat sequences [31-34], standing variation [35], sequencing and/or genome assembly errors [35, 36]. However, in all cases, G47ΔhIL12A consists of a replacement of the HCMV IE enhancer by HSV repeat sequences.

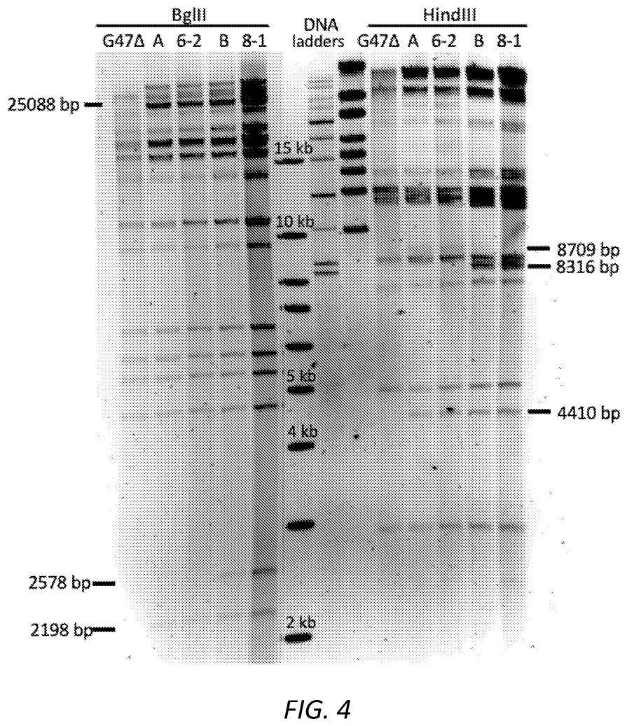

Restriction endonuclease digestion analyses of two high (A and 6-2-A8G1G6H4) and two low (B and 8-1-E2A11E11) hIL-12 level isolates (FIG. 4) confirmed the viral genome structure identified by sequencing, with both high and both low producers having the same fragment sizes, which were different between high and low isolates. The only restriction endonuclease digestion fragments with altered mobility/size compared to G47Δ were those in the ICP6 region of the virus genome that are indicated with their sizes (FIG. 4). This indicates that sequencing did not miss other potential deletions/insertions.

### Example 2. Characterization of G47ΔhIL12 Isolates

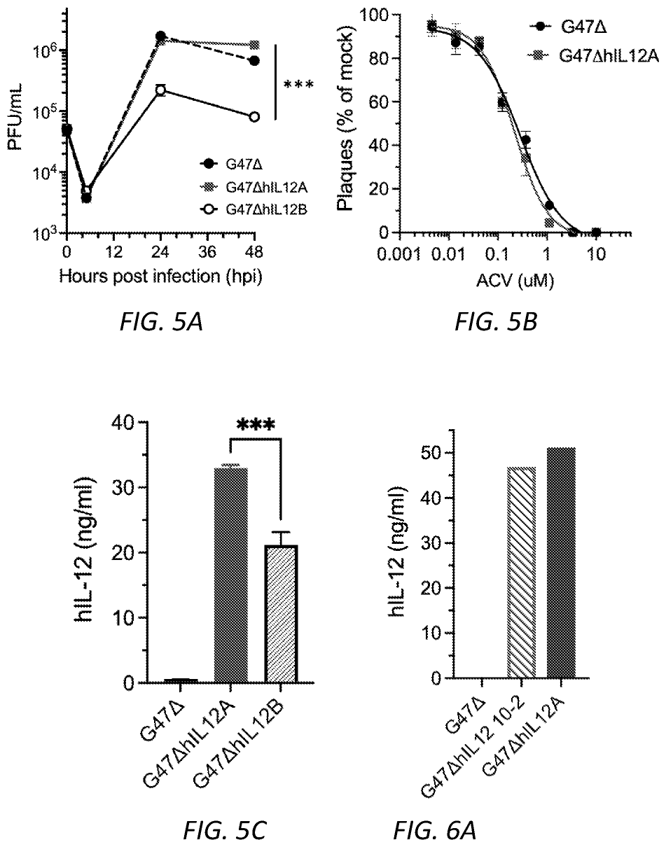

To investigate the effects of the hIL-12 cassette inserted into G47Δ on virus replication, we performed a single-step growth experiment comparing the two G47ΔhIL12 isolates (A and B) with parental G47Δ in Vero cells. By 24 hpi virus yield had reached a maximum and plateaued for all 3 viruses (FIG. 5A). However, the virus yield for G47ΔhIL12B was significantly less than for G47ΔhIL12A, which was similar to G47Δ (FIG. 5A). One important safety feature of oHSV is the availability of effective clinically approved anti-viral drugs like acyclovir. In addition, ICP6 mutants, like G47Δ, are hypersensitive to acyclovir [37]. To confirm that G47ΔhIL12A retained sensitivity to acyclovir, we performed a plaque reduction assay in Vero cells. Dose-response curves were fitted to the data and the IC50 values were determined. G47ΔhIL12A had a slightly, non-significant reduced IC50 (0.22 μM) compared to G47Δ (0.28 μM) (FIG. 5B). hIL-12 production was measured in infected Vero cell supernatants by ELISA. G47ΔhIL12A produced significantly more hIL-12 than G47ΔhIL12B at 24 hpi (FIG. 5C).

### Example 3. Expression of Biologically Active hIL12

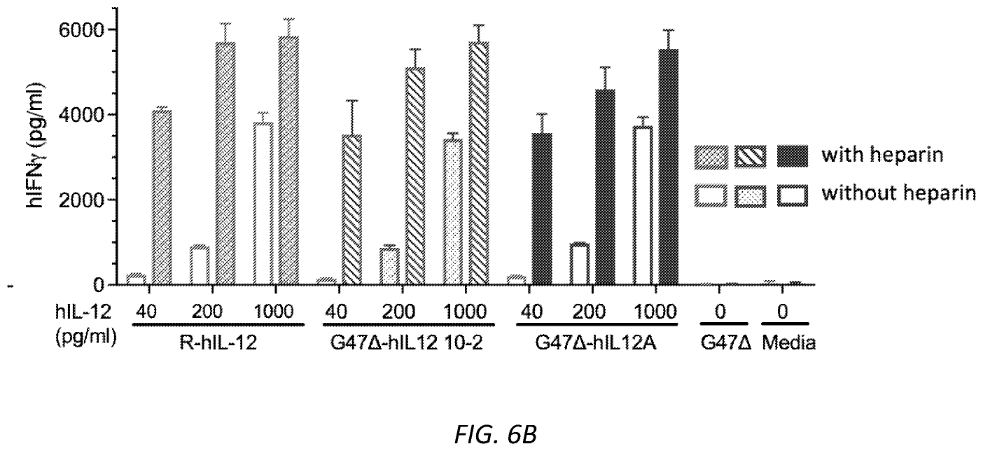

The biological activity of hIL-12 produced by G47ΔhIL12A was assessed with a functional assay using human NK cell line NK-92MI [38] and hIL-12 induced IFN-γ production [39]. Recombinant hIL-12 with known functional activity was used as a positive control for the induction of IFN-γ by NK-92MI cells (FIG. 6B). hIL-12, produced after infection of Vero cells with G47ΔhIL12A and 10-2-A1B5G3 (from FIG. 6A), or R-hIL12 was added to NK-92MI cells at indicated final concentrations with or without 10 μg/ml heparin. HIL-12-dependent secretion of IFN-γ into the supernatant of the NK-92MI cell culture was quantified by ELISA. NK-92MI cells were highly responsive to both R-hIL12 and virus-produced hIL-12, inducing IFN-γ production in a dose-dependent manner (FIG. 6B). HIL-12 is a heparin-binding cytokine, and its activity was previously shown to be enhanced by heparin as an immunomodulatory agent [40, 41]. Thus, we examined the effect of heparin on hIL-12-induced IFN-γ. Heparin greatly enhanced the activity of hIL12, especially at lower concentrations (FIG. 6B).

### Example 4. Growth and Cytotoxic Properties of G47ΔhIL12A Compared to Parental G47Δ in GSCs

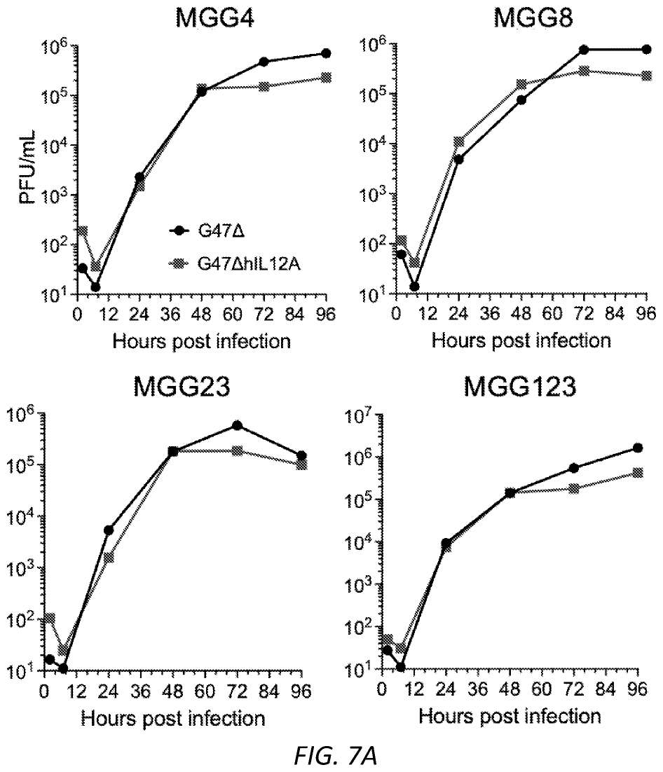

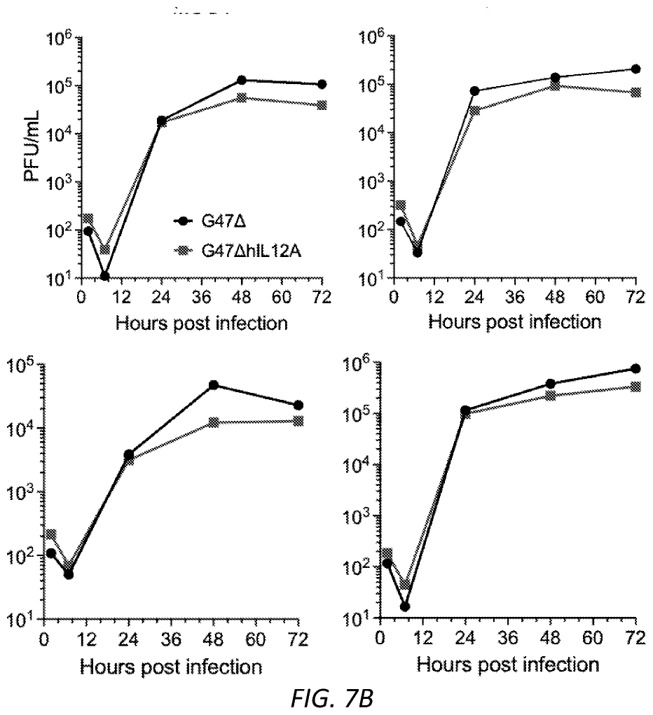

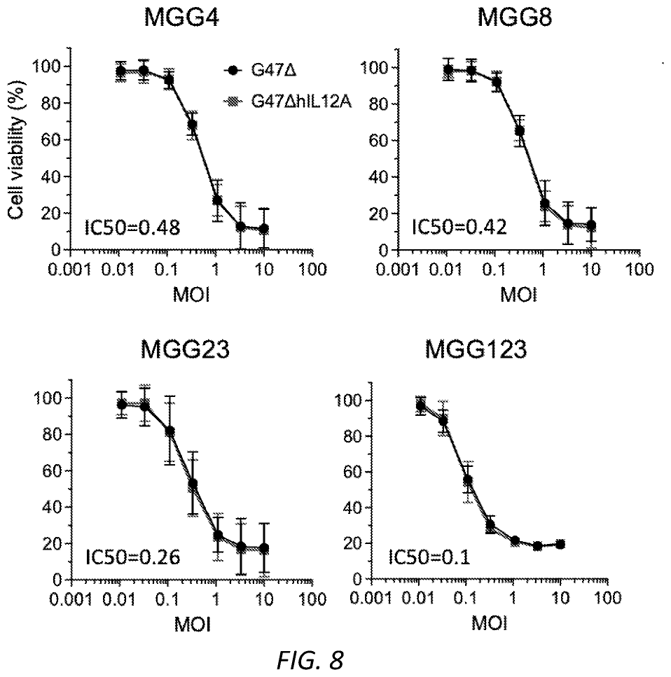

Virus growth and hIL-12 expression in permissive Vero cells enabled the selection and isolation of the recombinant virus isolates. For potential therapeutic purposes in cancer patients, G47ΔhIL12A must be active in human cancer cells. For glioblastoma, patient derived GSCs are rigorous and representative models for in vitro studies [6], as well as important therapeutic targets. We used a panel of four human GSC lines to compare the replication of G47ΔhIL12A with that of parental G47Δ. Dissociated GSCs were infected, supernatants harvested at the indicated time points, and the titers of infectious virus were determined by plaque assay on Vero cells. The growth of G47ΔhIL12A was similar to G47Δ in a multi-step growth experiment after infection at low MOI (0.3) (FIG. 7A) and a single-step growth experiment after infection with high MOI (1.5) virus (FIG. 7B). All GSCs tested were susceptible to G47ΔhIL12 replication. The oncolytic activity or cytotoxicity against the GSCs is an important determinant of oHSV efficacy. We next compared the cytotoxicity of G47ΔhIL12A and G47Δ against human GSCs. GSCs were infected with the indicated MOIs of G47Δ and G47ΔhIL12A, and cell viability determined after 4 days using an MTS assay. Similar to what was seen with virus replication, G47ΔhIL12A was very effective at inhibiting the growth of all GSCs tested in vitro (IC50=0.49, 0.42, 0.26, and 0.098 for MGG4, 8, 23, 123, respectively), the same as for G47Δ (FIG. 8).

### Example 5. Production of hIL-12 from Infected GSCs

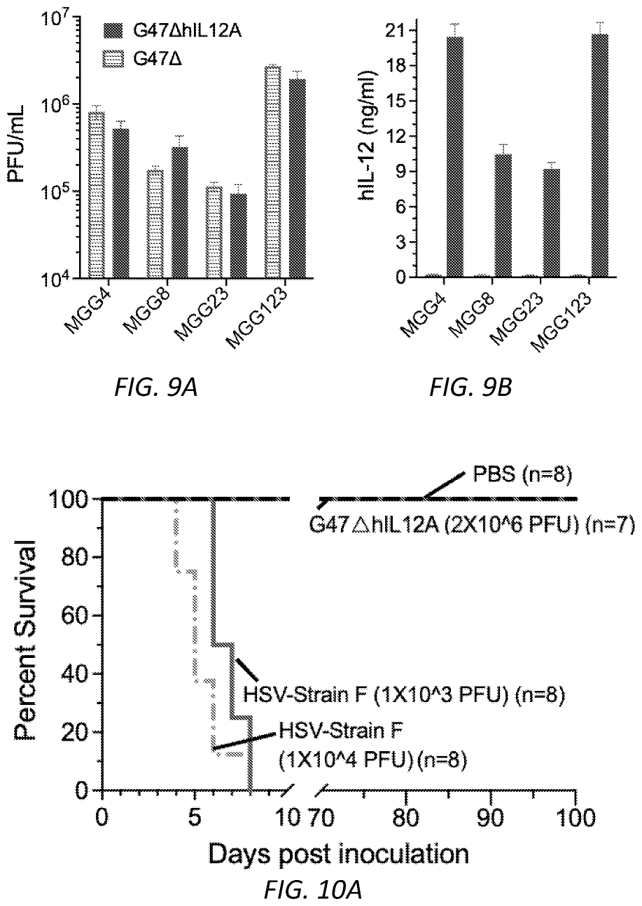

We next tested the production of hIL-12 from virus-infected GSCs. Human GSCs were infected with G47Δ and G47ΔhIL12A at MOI=1.5 for 30 hrs. Virus yield was determined by plaque assay (FIG. 9A) and hIL-12 levels were measured by ELISA (FIG. 9B). Virus yield in the four GSC lines varied, with MGG123 supporting the highest virus production and MG23 the lowest (FIG. 9B). MGG123 was also the most sensitive line to oHSV killing (FIG. 8). There was also variability in hIL-12 production in the different GSC lines, with MGG4 and MGG123 supporting the highest levels of hIL-12, and MGG23 the least. However, all GSC lines produced significant amounts of hIL-12, in a range from 9 to 20 ng/mL (FIG. 9B).

### Example 6. In Vivo Safety of G47ΔhIL12A after Intracerebral Injection in HSV-Sensitive Mice

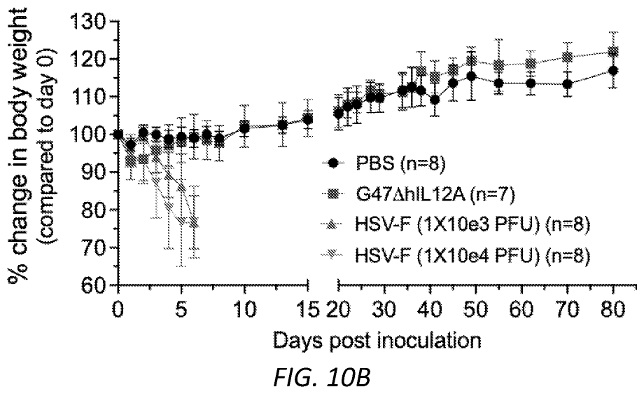

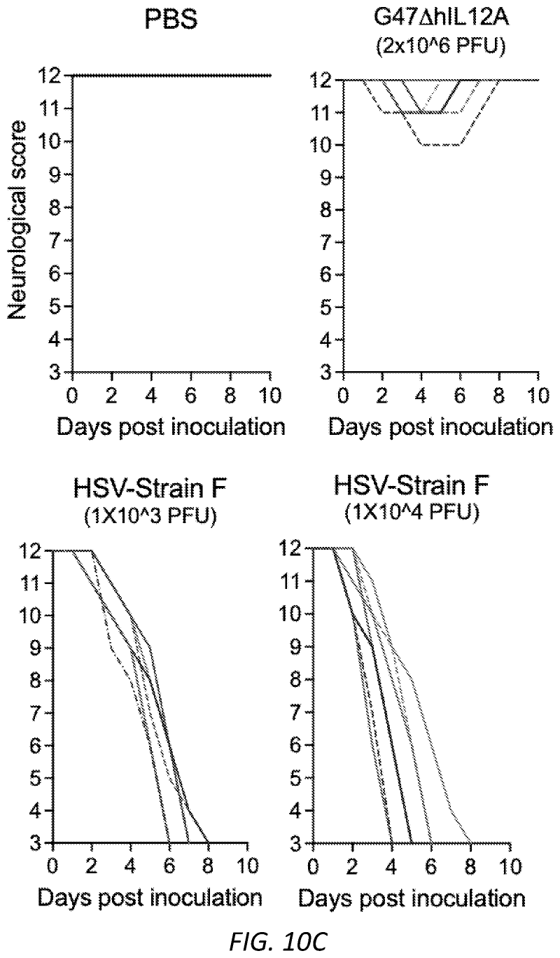

To bring the oncolytic virus into the clinic it is critical that oHSV is not neuropathogenic when delivered into the brain. To examine the safety of G47ΔhIL12A in the brain, HSV-1 susceptible BALB/c female mice were stereotactically inoculated (right striatum) with G47Δ-hIL-12 (2×10⁶ PFU), wild-type HSV-1 strain F (either 1×10² or 1×10³ PFU) or PBS, and followed for neurologic symptoms, weight loss and long-term survival (FIG. 10). All mice inoculated with as little as 1000 PFU of HSV-1 strain F became moribund within 8 days, whereas all mice inoculated with the highest obtainable dose of G47ΔhIL12A (2×10⁶ PFU) survived long term (FIG. 10A). G47ΔhIL12A inoculated mice showed a consistent increase in body weight as did PBS-inoculated mice after the first week of inoculation (FIG. 10B). We further quantified neurologic deficits using a scoring scale for morbidity, with all mice inoculated with HSV1 strain F showing severe symptoms within a week, while 6 of 7 mice inoculated with G47ΔhIL12A exhibited only very mild transient symptoms during the first week after virus inoculation (FIG. 10C), as seen previously with other oHSV, including those in the clinic [5, 42]. Histopathology confirmed HSV encephalitis in mice inoculated with wild-type strain F, including vascularization, Cowdry A inclusions, degraded ependymal cell layer of ventricles, and HSV-positive cells. Thus, G47ΔhIL12A is safe after intracerebral injection at a dose 20,000-fold higher than a lethal dose of wild-type HSV-1, which reflects the safety of G47Δ that has been in clinical trials in glioma patients in Japan without serious adverse events attributed to the virus [43]. hIL-12 expression from a different oHSV (M032) was safe after intracerebral injection in nonhuman primates [44], and safe and efficacious in dogs with sporadic glioma [45].

### REFERENCES

1. Martuza R L, Malick A, Markert J M, Ruffner K L, Coen D M. Experimental therapy of human glioma by means of a genetically engineered virus mutant. Science. 1991; 252:854-6.
2. Mineta T, Rabkin S D, Yazaki T, Hunter W D, Martuza R L. Attenuated multi-mutated herpes simplex virus-1 for the treatment of malignant gliomas. Nature Medicine. 1995; 1:938-43.
3. Markert J M, Medlock M D, Rabkin S D, Gillespie G Y, Todo T, Hunter W D, Palmer C A, Feigenbaum F, Tornatore C, Tufaro F, Martuza R L. Conditionally replicating herpes simplex virus mutant, G207 for the treatment of malignant glioma: results of a phase I trial. Gene Ther. 2000; 7(10):867-74.
4. Friedman G K, Johnston J M, Bag A K, Bernstock J D, Li R, Aban I, Kachurak K, Nan L, Kang K D, Totsch S, Schlappi C, Martin A M, Pastakia D, McNall-Knapp R, Farouk Sait S, Khakoo Y, Karajannis M A, Woodling K, Palmer J D, Osorio D S, Leonard J, Abdelbaki M S, Madan-Swain A, Atkinson T P, Whitley R J, Fiveash J B, Markert J M, Gillespie G Y. Oncolytic HSV-1 G207 Immunovirotherapy for Pediatric High-Grade Gliomas. N Engl J Med. 2021; 384(17):1613-22. doi: 10.1056/NEJMoa2024947.
5. Todo T, Martuza R L, Rabkin S D, Johnson P A. Oncolytic herpes simplex virus vector with enhanced MHC class I presentation and tumor cell killing. Proc Natl Acad Sci USA. 2001; 98(11):6396-401.
6. Wakimoto H, Kesari S, Farrell C J, Curry W T, Jr., Zaupa C, Aghi M, Kuroda T, Stemmer-Rachamimov A, Shah K, Liu T C, Jeyaretna D S, Debasitis J, Pruszak J, Martuza R L, Rabkin S D. Human glioblastoma-derived cancer stem cells: establishment of invasive glioma models and treatment with oncolytic herpes simplex virus vectors. Cancer Res. 2009; 69(8):3472-81. doi: 10.1158/0008-5472.Can-08-3886. PMCID: PMC2785462.
7. Peters C, Paget M, Tshilenge K-T, Saha D, Antoszczyk S, Baars A, Frost T, Martuza R L, Wakimoto H, Rabkin S D. Restriction of Replication of Oncolytic Herpes Simplex Virus with a Deletion of γ34.5 in Glioblastoma Stem-Like Cells. Journal of Virology. 2018; 92:e00246-18.
8. Todo T, Ino Y, Ohtsu H, Shibahara J, Tanaka M. A phase I/II study of triple-mutated oncolytic herpes virus G47Δ in patients with progressive glioblastoma. Nat Commun. 2022; 13(1):4119. doi: 10.1038/s41467-022-31262-y. PMCID: PMC9304402 therapeutic methods.” 4212897). All other authors have no conflict of interest to declare.
9. Todo T, Ito H, Ino Y, Ohtsu H, Ota Y, Shibahara J, Tanaka M. Intratumoral oncolytic herpes virus G47Δ for residual or recurrent glioblastoma: a phase 2 trial. Nat Med. 2022. doi: 10.1038/s41591-022-01897-x.
10. Daiichi-Sankyo. Daiichi Sankyo Launches DELYTACT® Oncolytic Virus G47Δ in Japan 2021. Available from: daiichisankyo.com/files/news/pressrelease/pdf/202111/20211101_E.pdf.
11. Saha D, Martuza R L, Rabkin S D. Macrophage Polarization Contributes to Glioblastoma Eradication by Combination Immunovirotherapy and Immune Checkpoint Blockade. Cancer Cell. 2017; 32(2):253-67. doi: 10.1016/j.ccell.2017.07.006. PMCID: PMC5568814.
12. Himes B T, Geiger P A, Ayasoufi K, Bhargav A G, Brown D A, Parney I F. Immunosuppression in Glioblastoma: Current Understanding and Therapeutic Implications. Front Oncol. 2021; 11:770561. doi: 10.3389/fonc.2021.770561. PMCID: PMC8581618.
13. Kanai R, Wakimoto H, Martuza R L, Rabkin S D. A Novel Oncolytic Herpes Simplex Virus that Synergizes with Phosphoinositide 3-kinase/Akt Pathway Inhibitors to Target Glioblastoma Stem Cells. Clin Cancer Res. 2011; 17(11):3686-96.
14. Boshart M, Weber F, Jahn G, Dorsch-Hasler K, Fleckenstein B, Schaffner W. A very strong enhancer is located upstream of an immediate early gene of human cytomegalovirus. Cell. 1985; 41(2):521-30. doi: 10.1016/s0092-8674(85)80025-8.
15. Wakimoto H, Mohapatra G, Kanai R, Curry W T, Jr., Yip S, Nitta M, Patel A P, Barnard Z R, Stemmer-Rachamimov A O, Louis D N, Martuza R L, Rabkin S D. Maintenance of primary tumor phenotype and genotype in glioblastoma stem cells. Neuro Oncol. 2012; 14(2):132-44. doi: 10.1093/neuonc/norl95. PMCID: 3266381.
16. Nigim F, Cavanaugh J, Patel A P, Curry W T, Jr., Esaki S, Kasper E M, Chi A S, Louis D N, Martuza R L, Rabkin S D, Wakimoto H. Targeting Hypoxia-Inducible Factor 1alpha in a New Orthotopic Model of Glioblastoma Recapitulating the Hypoxic Tumor Microenvironment. J Neuropathol Exp Neurol. 2015; 74(7):710-22. doi: 10.1097/nen.0000000000000210. PMCID: 4473779.
17. Cheema T A, Wakimoto H, Fecci P E, Ning J, Kuroda T, Jeyaretna D S, Martuza R L, Rabkin S D. Multifaceted oncolytic virus therapy for glioblastoma in an immunocompetent cancer stem cell model. Proc Natl Acad Sci USA. 2013; 110(29):12006-11. doi: 10.1073/pnas.1307935110. PMCID: 3718117.
18. Rampling R, Cruickshank G, Papanastassiou V, Nicoll J, Hadley D, Brennan D, Petty R, MacLean A, Harland J, McKie E, Mabbs R, Brown M. Toxicity evaluation of replication-competent herpes simplex virus (ICP 34.5 null mutant 1716) in patients with recurrent malignant glioma. Gene Ther. 2000; 7(10):859-66.
19. Patel D M, Foreman P M, Nabors L B, Riley K O, Gillespie G Y, Markert J M. Design of a Phase I Clinical Trial to Evaluate M032, a Genetically Engineered HSV-1 Expressing IL-12, in Patients with Recurrent/Progressive Glioblastoma Multiforme, Anaplastic Astrocytoma, or Gliosarcoma. Human gene therapy Clinical development. 2016; 27(2):69-78. doi: 10.1089/humc.2016.031. PMCID: PMC4932657.
20. Kanai R, Zaupa C, Sgubin D, Antoszczyk S J, Martuza R L, Wakimoto H, Rabkin S D. Effect of gamma34.5 deletions on oncolytic herpes simplex virus activity in brain tumors. J Virol. 2012; 86(8):4420-31. doi: 10.1128/jvi.00017-12. PMCID: 3318611.
21. Clarke M E. Clinical and Therapeutic Implications of Cancer Stem Cells. N Engl J Med. 2019; 380(23):2237-45. doi: 10.1056/NEJMra1804280.
22. Toda M, Martuza R L, Rabkin S D. Combination suicide/cytokine gene therapy as adjuvants to a defective herpes simplex virus-based cancer vaccine. Gene Ther. 2001; 8(4):332-9.
23. Todo T, Martuza R L, Dallman M J, Rabkin S D. In situ expression of soluble B7-1 in the context of oncolytic herpes simplex virus induces potent antitumor immunity. Cancer Res. 2001; 61(1):153-61.
24. Liu T C, Zhang T, Fukuhara H, Kuroda T, Todo T, Canron X, Bikfalvi A, Martuza R L, Kurtz A, Rabkin S D. Dominant-negative fibroblast growth factor receptor expression enhances antitumoral potency of oncolytic herpes simplex virus in neural tumors. Clin Cancer Res. 2006; 12(22):6791-9.
25. Han Z Q, Assenberg M, Liu B L, Wang Y B, Simpson G, Thomas S, Coffin R S. Development of a second-generation oncolytic Herpes simplex virus expressing TNFalpha for cancer therapy. J Gene Med. 2007; 9(2):99-106.
26. Passaro C, Alayo Q, De Laura I, McNulty J, Grauwet K, Ito H, Bhaskaran V, Mineo M, Lawler S E, Shah K, Speranza M C, Goins W, McLaughlin E, Fernandez S, Reardon D A, Freeman G J, Chiocca E A, Nakashima H. Arming an Oncolytic Herpes Simplex Virus Type 1 with a Single-chain Fragment Variable Antibody against PD-1 for Experimental Glioblastoma Therapy. Clin Cancer Res. 2019; 25(1):290-9. doi: 10.1158/1078-0432.Ccr-18-2311. PMCID: PMC6800097.
27. Goldstein D J, Weller S K. Herpes simplex virus type 1-induced ribonucleotide reductase activity is dispensible for virus growth and DNA synthesis: Isolation and characterization of an ICP6 lacZ insertion mutant. J Virol. 1988; 62:196-205.
28. Kintner R L, Brandt C R. Rapid small-scale isolation of herpes simplex virus DNA. J Virol Methods. 1994; 48(2-3):189-96. doi: 10.1016/0166-0934(94)90118-x.
29. Chou J, Kern E R, Whitley R J, Roizman B. Mapping of herpes simplex virus-1 neurovirulence to gamma 34.5, a gene nonessential for growth in culture. Science. 1990; 250:1262-6.
30. López-Muñoz A D, Rastrojo A, Kropp K A, Viejo-Borbolla A, Alcami A. Combination of long- and short-read sequencing fully resolves complex repeats of herpes simplex virus 2 strain MS complete genome. Microb Genom. 2021; 7(6). doi: 10.1099/mgen.0.000586. PMCID: PMC8461477.
31. Tognon M, Cassai E, Rotola A, Roizman B. The heterogenous regions in herpes simplex virus 1 DNA. Microbiologica. 1983; 6(3):191-8.
32. Wagner M J, Summers W C. Structure of the joint region and the termini of the DNA of herpes simplex virus type 1. J Virol. 1978; 27(2):374-87. doi: 10.1128/jvi.27.2.374-387.1978. PMCID: PMC354176.
33. Davison A J, Wilkie N M. Nucleotide sequences of the joint between the L and S segments of herpes simplex virus types 1 and 2. J Gen Virol. 1981; 55 (Pt 2):315-31. doi: 10.1099/0022-1317-55-2-315.
34. Umene K. Recombination of the internal direct repeat element DR2 responsible for the fluidity of the a sequence of herpes simplex virus type 1. J Virol. 1991; 65(10):5410-6. doi: 10.1128/jvi.65.10.5410-5416.1991. PMCID: PMC249022.
35. Kuny C V, Szpara M L. Alphaherpesvirus Genomics: Past, Present and Future. Curr Issues Mol Biol. 2021; 42:41-80. doi: 10.21775/cimb.042.041. PMCID: PMC7946737.
36. Al Qaffas A, Nichols J, Davison A J, Ourahmane A, Hertel L, McVoy M A, Camiolo S. LoReTTA, a user-friendly tool for assembling viral genomes from PacBio sequence data. Virus Evol. 2021; 7(1):veab042. doi: 10.1093/ve/veab042. PMCID: PMC8111061.
37. Coen D M, Goldstein D J, Weller S K. Herpes simplex virus ribonucleotide reductase mutants are hypersensitive to acyclovir Antimicrob Agents Chemother. 1989; 33(8):1395-9.
38. Tam Y K, Maki G, Miyagawa B, Hennemann B, Tonn T, Klingemann H G. Characterization of genetically altered, interleukin 2-independent natural killer cell lines suitable for adoptive cellular immunotherapy. Hum Gene Ther. 1999; 10(8):1359-73. doi: 10.1089/10430349950018030.
39. Cheng M, Fei B, Zheng X, Chen Y, Sun R, Wei H, Tian Z. A new reliable bioassay for determining the biological activity of human interleukin-12 by using human NK cell line NKG cells. Int Immunopharmacol. 2012; 13(1):109-13. doi: 10.1016/j.intimp.2012.03.015.
40. Nguyen K G, Gillam F B, Hopkins J J, Jayanthi S, Gundampati R K, Su G, Bear J, Pilkington G R, Jalah R, Felber B K, Liu J, Thallapuranam S K, Zaharoff D A. Molecular mechanisms of heparin-induced modulation of human interleukin 12 bioactivity. J Biol Chem. 2019; 294(12):4412-24. doi: 10.1074/jbc.RA118.006193. PMCID: PMC6433073.
41. Jayanthi S, Koppolu B P, Nguyen K G, Smith S G, Felber B K, Kumar T K S, Zaharoff D A. Modulation of Interleukin-12 activity in the presence of heparin. Sci Rep. 2017; 7(1):5360. doi: 10.1038/s41598-017-05382-1. PMCID: PMC5509706.
42. Sundaresan P, Hunter W D, Martuza R L, Rabkin S D. Attenuated, replication-competent herpes simplex virus type 1 mutant G207: safety evaluation in mice. J Virol. 2000; 74(8):3832-41.
43. PMDA. PMDA drug review—Delytact 2021. Available from: pmda.go.jp/english/review-services/reviews/approved-information/0004.html.
44. Roth J C, Cassady K A, Cody J J, Parker J N, Price K H, Coleman J M, Peggins J O, Noker P E, Powers N W, Grimes S D, Carroll S L, Gillespie G Y, Whitley R J, Markert J M. Evaluation of the safety and biodistribution of M032, an attenuated herpes simplex virus type 1 expressing hIL-12, after intracerebral administration to aotus nonhuman primates. Human gene therapy Clinical development. 2014; 25(1):16-27. doi: 10.1089/humc.2013.201. PMCID: PMC4047998.
45. Omar N B, Bentley R T, Crossman D K, Foote J B, Koehler J W, Markert J M, Platt S R, Rissi D R, Shores A, Sorjonen D, Yanke A B, Gillespie G Y, Chambers M R. Safety and interim survival data after intracranial administration of M032, a genetically engineered oncolytic HSV-1 expressing IL-12, in pet dogs with sporadic gliomas. Neurosurg Focus. 2021; 50(2):E5. doi: 10.3171/2020.11. Focus20844. PMCID: PMC8383155.
46. Lee E Q. Immune checkpoint inhibitors in GBM. J Neurooncol. 2021; 155(1):1-11. doi: 10.1007/s11060-021-03859-8.
47. Chiocca E A, Rabkin S D. Oncolytic viruses and their application to cancer immunotherapy. Cancer immunology research. 2014; 2(4):295-300. Epub 2014/04/26. doi: 10.1158/2326-6066.CIR-14-0015. PubMed PMID: 24764576; PubMed Central PMCID: PMC4303349.
48. Lemos de Matos A, Franco L S, McFadden G. Oncolytic Viruses and the Immune System: The Dynamic Duo. Molecular therapy Methods & clinical development. 2020; 17:349-58. Epub 2020/02/20. doi: 10.1016/j.omtm.2020.01.001. PubMed PMID: 32071927; PubMed Central PMCID: PMC7015832
49. Jahan N, Ghouse S M, Martuza R L, Rabkin S D. In Situ Cancer Vaccination and Immunovirotherapy Using Oncolytic HSV. Viruses. 2021; 13(9). Epub 2021/09/29. doi: 10.3390/v13091740. PubMed PMID: 34578321; PubMed Central PMCID: PMC8473045.
50. Toda M, Martuza R L, Kojima H, Rabkin S D. In situ cancer vaccination: an IL-12 defective vector/replication-competent herpes simplex virus combination induces local and systemic antitumor activity. Journal of immunology. 1998; 160(9):4457-64. Epub 1998/05/09. PubMed PMID: 9574551.
51. Toda M, Rabkin S D, Kojima H, Martuza R L. Herpes simplex virus as an in situ cancer vaccine for the induction of specific anti-tumor immunity. Human gene therapy. 1999; 10(3):385-93. Epub 1999/02/27. doi: 10.1089/10430349950018832. PubMed PMID: 10048391.
52. de Graaf J F, de Vor L, Fouchier RAM, van den Hoogen B G. Armed oncolytic viruses: A kick-start for anti-tumor immunity. Cytokine & growth factor reviews. 2018; 41:28-39. Epub 2018/03/27. doi: 10.1016/j.cytogfr.2018.03.006. PubMed PMID: 29576283; PubMed Central PMCID: PMC7108398.
53. Zhang Y, Li Y, Chen K, Qian L, Wang P. Oncolytic virotherapy reverses the immunosuppressive tumor microenvironment and its potential in combination with immunotherapy. Cancer cell international. 2021; 21(1):262. Epub 2021/05/15. doi: 10.1186/s12935-021-01972-2. PubMed PMID: 33985527; PubMed Central PMCID: PMC8120729.
54. Nguyen H M, Sah N, Humphrey M R M, Rabkin S D, Saha D. Growth, Purification, and Titration of Oncolytic Herpes Simplex Virus. Journal of visualized experiments: JoVE. 2021(171). Epub 2021/06/01. doi: 10.3791/62677. PubMed PMID: 34057449; PubMed Central PMCID: PMC8447238
55. Chou J, Roizman B. The herpes simplex virus 1 gene for ICP34.5, which maps in inverted repeats, is conserved in several limited-passage isolates but not in strain 17syn+. Journal of virology. 1990; 64(3):1014-20. Epub 1990/03/01. doi: 10.1128/JVI.64.3.1014-1020.1990. PubMed PMID: 2154589; PubMed Central PMCID: PMC249211.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An expression cassette comprising an enhancer sequence comprising two or more DR2 repeat sequence units (CGCTCCTCCCCC (SEQ ID NO:7) inserted upstream of a promoter, preferably within 500, 250, 200, or 100 nucleotides of the promoter, and optionally a transgene operably linked to the promoter.

2. The expression cassette of claim 1, wherein the enhancer sequence comprises from six to 24 DR2 repeat sequence units (CGCTCCTCCCCC (SEQ ID NO:7).

3. The expression cassette of claim 1, wherein the enhancer sequence comprises:
(SEQ ID NO: 4)
CTCCCCCCGCTCCTCCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCTCCCCCCCCCGCTCCCGCGGCCCCGC
CCCCAACGCCCGCTCCTCCCCCCGCTCCCGCGGCCCCGCCC,
or
(SEQ ID NO: 5)
CTCCCCCCGCTCCTCCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCTCCCCCCCCCGCTCCCGCGGCCCCGC
CCCCAACGCCCGCTCCTCCCCCCGCTCCCGCGGCCCCGCCCCCAA
CGCCCGCCGCGCGCGCGCACGCCGCCCTTGACTCACGGGGATTTC
CAAGTCTCCACCCCATTGACGTC,
or
(SEQ ID NO: 15)
ATCCCCCGCTCCTCCCCCGCTCCTCCCCCCGCTCCTCCCCCCGCT
CCTCCCCCCGCTCCTCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCCGCGGCCCCGCCCCCAACGCCCGCTC
CTCCCCCCGCTCCCGCGGCCCCGCCCCCAACGCCCGCCGCGCGCG
CGCACGCCGCCCGGACCGCCGCCCGCCTTTTTTGCGCGCCGCCCC
GCCCGCGGGGGGCCCGGGCTGCGCCGCCGCGCTTTAAAGGGCCGC
GCGCGACCCCCGGGGGGTGTGTTTCGGGGGGGGCCCGTTTCTCCC
GCTCCTCCCCCCGCTCCTCCCCCCGCTCCTCCCCCCGCTCCTCCC
CCCGCTCCTCCCCCCGCTCCTCCCCCCGCTCCTCCCCCCGCTCCT
CCCCCCGCTCCTCCCCCCGCTCCTCCCCCCGCTCCTCCCCCCGCT
CCCGCGGCCCCGCCCCCAACGCCCGCTCCTCCCCCCGCTCCCGCG
GCCCCGCCCCCAACGCCCGCCGCGCGCGCGCACGCCGCCCT.

4. The expression cassette of claim 1, wherein the expression cassette is in a plasmid, cosmid, or viral vector.

5. The expression cassette of claim 4, wherein the viral vector is selected from the group consisting of recombinant retroviruses, adenovirus, adeno-associated virus, alphavirus, oncoviruses, and lentiviruses.

6. The expression cassette of claim 5, wherein the oncovirus is a herpes simplex virus.

7. The expression cassette of claim 5, wherein the herpes simplex virus comprises one or more of: one or more alterations selected from deletion of both copies of γ34.5; deletion of ICP47 and/or Us11 promoter; an inactivating insertion in ICP6; downregulation, tumor-selective expression, and/or specific amino acid mutations of γ34.5, ICP47, and/or ICP6; upregulation or tumor-selective expression of Us11; and a reporter gene like GFP or LacZ.

8. The expression cassette of claim 5, which is in G207, MG18L, Δ68H-6, or G47Δ.

9. The expression cassette of claim 1 comprising a transgene, optionally wherein the transgene is selected from cytokines, chemokines, complement components and their receptors, immune system accessory molecules, adhesion molecules, adhesion receptor molecules, and adenosine-degrading enzymes.

10. The expression cassette of claim 9, wherein the cytokine is selected from interleukins, optionally any of interleukins 1-15, preferably IL-12; α, β, or γ-interferons; tumor necrosis factor; granulocyte macrophage colony stimulating factor (GM-CSF); macrophage colony stimulating factor (M-CSF); and granulocyte colony stimulating factor (G-CSF).

11. The expression cassette of claim 9, wherein the chemokine is selected from B7.1 and B7.2.

12. The expression cassette of claim 9, wherein the adhesion molecule is selected from ICAM-1, 2, and 3.

13. The expression cassette of claim 9, wherein the immune system accessory molecule is selected from neutrophil activating protein (NAP), macrophage chemoattractant and activating factor (MCAF), RANTES, and macrophage inflammatory peptides MTP-1a and MTP-1b.

14. A method of expressing a transgene in a cell, the method comprising contacting the cell with the expression cassette of claim 1, wherein the expression cassette comprises the transgene.

15. An oncolytic herpes simplex virus (oHSV) comprising a promoter and an enhancer sequence, wherein the enhancer sequence comprises from six to 24 DR2 repeat sequence units (CGCTCCTCCCCC (SEQ ID NO:7), preferably wherein the enhancer sequence comprises:
(SEQ ID NO: 4)
CTCCCCCCGCTCCTCCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCTCCCCCCCCCGCTCCCGCGGCCCCGC
CCCCAACGCCCGCTCCTCCCCCCGCTCCCGCGGCCCCGCCC,
or
(SEQ ID NO: 5)
CTCCCCCCGCTCCTCCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCTCCCCCCCCCGCTCCCGCGGCCCCGC
CCCCAACGCCCGCTCCTCCCCCCGCTCCCGCGGCCCCGCCCCCAA
CGCCCGCCGCGCGCGCGCACGCCGCCCTTGACTCACGGGGATTTC
CAAGTCTCCACCCCATTGACGTC,
or
(SEQ ID NO: 15)
ATCCCCCGCTCCTCCCCCGCTCCTCCCCCCGCTCCTCCCCCCGCT
CCTCCCCCCGCTCCTCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCCGCGGCCCCGCCCCCAACGCCCGCTC
CTCCCCCCGCTCCCGCGGCCCCGCCCCCAACGCCCGCCGCGCGCG
CGCACGCCGCCCGGACCGCCGCCCGCCTTTTTTGCGCGCCGCCCC
GCCCGCGGGGGGCCCGGGCTGCGCCGCCGCGCTTTAAAGGGCCGC
GCGCGACCCCCGGGGGGTGTGTTTCGGGGGGGGCCCGTTTCTCCC
GCTCCTCCCCCCGCTCCTCCCCCCGCTCCTCCCCCCGCTCCTCCC
CCCGCTCCTCCCCCCGCTCCTCCCCCCGCTCCTCCCCCCGCTCCT
CCCCCCGCTCCTCCCCCCGCTCCTCCCCCCGCTCCTCCCCCCGCT
CCCGCGGCCCCGCCCCCAACGCCCGCTCCTCCCCCCGCTCCCGCG
GCCCCGCCCCCAACGCCCGCCGCGCGCGCGCACGCCGCCCT.

16. The oHSV of claim 15, further comprising a transgene, optionally wherein the transgene is selected from cytokines, chemokines, complement components and their receptors, immune system accessory molecules, adhesion molecules, adhesion receptor molecules, and adenosine-degrading enzymes.

17. The oHSV of claim 16, wherein the cytokine is selected from interleukins, optionally any of interleukins 1-15; α, β, or γ-interferons; tumor necrosis factor; granulocyte macrophage colony stimulating factor (GM-CSF); macrophage colony stimulating factor (M-CSF); and granulocyte colony stimulating factor (G-CSF).

18. The oHSV of claim 17, wherein the transgene is human interleukin 12 (hIL-12).

19. The oHSV of claim 16, wherein the chemokine is selected from B7.1 and B7.2.

20. The oHSV of claim 16, wherein the adhesion molecule is selected from ICAM-1, -2, and -3.

21. The oHSV of claim 16, wherein the immune system accessory molecule is selected from neutrophil activating protein (NAP), macrophage chemoattractant and activating factor (MCAF), RANTES, and macrophage inflammatory peptides MTP-1a and MTP-1b.

22. The oHSV of claim 16, comprising (i) a mutation that consists of a deletion of a region corresponding to the BstEII-EcoNI fragment of the BamHI x fragment of F strain of herpes simplex virus I, and (ii) an inactivating mutation in a γ34.5 neurovirulence locus, and optionally (iii) optionally an inactivating mutation elsewhere in the genome and/or (iv) a reporter gene like GFP or LacZ.

23. The oHSV of claim 22, wherein the oHSV is G47Δ.

24. An oncolytic herpes simplex virus (oHSV) comprising a promoter and an enhancer sequence comprising:
(SEQ ID NO: 16)
CTCCCCCCGCTCCTCCCCCCGCTCCTCCCCCCGCTCCTCCCCCCG
CTCCTCCCCCCGCTCCTCCCCCCGCTCCTCCCCCCGCTCCTCCCC
CCGCTCCTCCCCCCGCTCCTCCCCCCCCCGCTCCCGCGGCCCCGC
CCCCAACGCCCGCTCCTCCCCCCGCTCCCGCGGCCCCGCCCCCAA
CGCCCGCCGCGCGCGCGCACGCCGCCCTTGACTCACGGGGATTTC
CAAGTCTCCACCCCATTGACGTCAATGGGAGTTTGTTTTGGCACC
AAAATCAACGGGACTTTCCAAAATGTCGTAACAACTCCGCCCCAT
TGACGCAAATGGGCGGTAGGCGTGTACGGTGGGAGGTCTATATAA
GCAGAGCTCTCTGGCTAACTAGAGAACCCACTGCTTACTGGCTTA
TCGAAATTAATACGACTCACTATAGGGAGACCCAAGCTTACCGGC
GAAGGAGGGCCACC.

25. The oHSV of claim 24, comprising (i) a mutation that consists of a deletion of a region corresponding to the BstEII-EcoNI fragment of the BamHI x fragment of F strain of herpes simplex virus I, and (ii) an inactivating mutation in a γ 34.5 neurovirulence locus, and optionally (iii) optionally an inactivating mutation elsewhere in the genome and/or (iv) a reporter gene like GFP or LacZ.

26. The oHSV of claim 25, wherein the oHSV is G47Δ.

27. The oHSV of claim 24, further comprising a human IL-12 transgene operably linked to the promoter and enhancer of SEQ ID NO:12.

28. An oHSV comprising a human IL-12 expression cassette comprising SEQ ID NO:17, optionally wherein the oHSV is G47Δ.

29. A method of treating a cancer in a subject, the method comprising administering to the subject an effective amount of the oHSV of claim 15.

30. The method of claim 29, wherein the cancer is a solid tumor.

31. The method of claim 30, wherein the solid tumor is a nervous system tumor, optionally an astrocytoma, oligodendroglioma, meningioma, neurofibroma, glioma, glioblastoma, ependymoma, schwannoma, neurofibrosarcoma, neuroblastoma, pituitary tumor, or medulloblastoma.

32. The method of claim 31, wherein the nervous system tumor is a glioblastoma.

33. The method of claim 32, wherein the solid tumor is melanoma, prostate carcinoma, renal cell carcinoma, pancreatic cancer, breast cancer, lung cancer, colon cancer, gastric cancer, fibrosarcoma, squamous cell carcinoma, neurectodermal, thyroid tumor, lymphoma, hepatoma, mesothelioma, or epidermoid carcinoma cells.

34. The method of claim 33, wherein the breast cancer is triple negative breast cancer.

35. The method of claim 29, further comprising administering an immunomodulator, optionally comprising one or more immune checkpoint inhibitors (ICIs) and/or inhibitors of the adenosine pathway.

36. The method of claim 35, wherein the one or more ICIs are selected from an antibody that binds to PD-1, CD40, PD-L1, Tim3, Lag3, CTLA-4, or T-cell immunoglobulin and ITIM domains (TIGIT).

37. The method of claim 36, wherein the one or more ICIs comprise an anti-PD-1 antibody and an anti-CTLA-4 antibody.

38. A method of treating glioblastoma in a subject, the method comprising administering to the subject a G47Δ oncolytic herpes simplex virus (oHSV) comprising a promoter and an enhancer sequence, wherein the enhancer sequence comprises from six to 24 DR2 repeat sequence units (CGCTCCTCCCCC (SEQ ID NO:7), preferably wherein the enhancer and promoter sequence comprises SEQ ID NO:16, in combination with an anti-PD-1 antibody and an anti-CTLA-4 antibody.

39. The method of claim 38, wherein the oHSV comprises SEQ ID NO:1, or a sequence at least 80%, 90%, 95%, or 99% identical to nucleotides 93-2490 of SEQ ID NO:1, or to nucleotides 416-2490 of SEQ ID NO:1.

40. The method of claim 39, wherein the oHSV is G47ΔhIL12A.

41. A method of treating glioblastoma in a subject, the method comprising administering to the subject an oncolytic herpes simplex virus (oHSV) comprising a promoter and an enhancer sequence, wherein the enhancer sequence comprises from six to 24 DR2 repeat sequence units (CGCTCCTCCCCC (SEQ ID NO:7), preferably wherein the enhancer and promoter sequence comprises SEQ ID NO:16, in combination with an anti-PD-1 antibody and an anti-CTLA-4 antibody.

42. The method of claim 40, wherein the oHSV is G207, G207-Us11, Δ68H-6, or MG18L.

43. The method of claim 40, wherein the oHSV comprises SEQ ID NO:1, or a sequence at least 80%, 90%, 95%, or 99% identical to nucleotides 93-2490 of SEQ ID NO:1, or to nucleotides 416-2490 of SEQ ID NO:1.
